(19) United States
(12) Patent Application Publication
(10) Pub. No.: US 2016/0143625 A1
(43) Pub. Date: May 26, 2016

(54) ULTRASONIC PROBE AND ULTRASONIC DIAGNOSIS APPARATUS

(71) Applicants: KABUSHIKI KAISHA TOSHIBA, Minato-Ku (JP); TOSHIBA MEDICAL SYSTEMS CORPORATION, Otawara-Shi (JP)

(72) Inventor: Hiroyuki SHIKATA, Nasushiobara (JP)

(73) Assignees: KABUSHIKI KAISHA TOSHIBA, Minato-Ku (JP); TOSHIBA MEDICAL SYSTEMS CORPORATION, Otawara-Shi (JP)

(21) Appl. No.: 14/948,770

(22) Filed: Nov. 23, 2015

(30) Foreign Application Priority Data
Nov. 26, 2014 (JP) 2014-238641
Nov. 19, 2015 (JP) 2015-226812

(51) Int. Cl. A61B 8/08; A61B 8/12; A61B 8/13; A61B 8/00

(52) U.S. Cl. CPC A61B 8/5207; A61B 8/4477; A61B 8/485; A61B 8/12; A61B 8/54; A61B 8/463; A61B 8/13

(57) ABSTRACT

An ultrasonic probe according to a present embodiment includes: at least one first transducer functioning as a transducer for excitation for executing excitation by an acoustic radiation pressure in an elastography mode; and second transducers functioning as transducers for detection for detecting a shear wave generated by the excitation in the elastography mode.

### CROSS-REFERENCE TO RELATED APPLICATION

This application is based upon and claims the benefit of priority from Japanese Patent Application No. 2014-238641, filed on Nov. 26, 2014, and Japanese Patent Application No. 2015-226812, filed on Nov. 19, 2015, the entire contents of which are incorporated herein by reference.

### FIELD

An embodiment as one aspect of the present invention relates to an ultrasonic probe and an ultrasonic diagnosis apparatus for transmitting/receiving ultrasonic waves.

### BACKGROUND

As a diagnosing method for a breast cancer, a hepatic cirrhosis, a vascular disorder and the like, a method (elastography) for quantifying and visualizing hardness of a tissue such as an organ in a living body from an ultrasonic echo signal instead of palpation by a doctor is known. The elastography is roughly classified into strain detecting elastography and acoustic radiation elastography. The strain detecting elastography presses and releases a body surface from outside the body and quantifies and visualizes relative hardness with respect to peripheral tissues from deformation (strain) of the organ caused by movement of the organ such as a spontaneously working heart and the like.

The acoustic radiation elastography is to transmit ultrasonic waves for excitation having relatively large energy generating an acoustic radiation pressure to a tissue of an organ in a living body and the like from outside the body. The acoustic radiation elastography is to quantify and visualize hardness (modulus of elasticity) of a tissue by calculating a sound speed of a shear wave generated as a lateral wave around the tissue by displacement (vibration) of the tissue.

Among them, in the acoustic radiation elastography, first, a tissue present at an excitation position is displaced by formation of an ultrasonic beam (excitation beam) for excitation by using an ultrasonic transducer unit for a B mode of an ultrasonic probe. Subsequently, by forming an ultrasonic beam (detection beam) for detection at a detection position around the excitation position by using the same ultrasonic transducer unit, a wave crest of the shear wave generated by the displacement of the tissue is detected by a tissue Doppler method or the like.

Then, in the acoustic radiation elastography, by counting traveling time from transmission time of the excitation beam to arrival time at the detection position of the wave crest of the shear wave, a sound speed of the shear wave from the excitation position to the detection position is calculated. Moreover, an average sound speed of the shear wave on the basis of sound speeds from the excitation position to the detection positions is calculated and a relative value of each sound speed to the average sound speed is calculated as information indicating hardness of the tissue.

Since a living body has viscosity, the wave crest of the shear wave becomes dull as it goes away from the excitation position. As a result, according to the prior-art technology, detection accuracy of the wave crest of the shear wave lowers at the detection position away from the excitation position and thus, uniformity of an image quality of the entire elastography image deteriorates.

Thus, in the prior-art technology, such processing is executed that a display range is divided into multiple blocks, and multiple detection positions (blocks) with high detection accuracy of the shear wave are connected so as to generate one sheet of an elastography image. For this purpose, multiple transmission sequences (combination of transmission of a series of excitation pulses and transmission of a series of detection pulses) need to be performed in correspondence with the multiple detection positions and thus, a frame rate of the elastography image and the like lowers for a portion of time required for the multiple transmission sequences. On the other hand, if the number of detection positions is decreased in order to maintain the frame rate, uniformity of the image quality of the elastography image lowers.

Moreover, if the frame rate of the elastography image and the like lowers, real-time properties are lost, and trouble that artifact is generated in the image by movement of the tissue in the living body also occurs.

The problem that the present invention is going to solve is to provide an ultrasonic probe and an ultrasonic diagnosis apparatus being able to generate information to generate an elastography image in time required for the minimum number of times of transmission sequences.

### BRIEF DESCRIPTION OF THE DRAWINGS

In accompanying drawings,

FIG. 1 is a schematic view illustrating constitution of an ultrasonic probe and an ultrasonic diagnosis apparatus according to a present embodiment;

FIG. 2 is a perspective view illustrating an appearance structure in a prior-art ultrasonic probe;

FIG. 3 is a view illustrating a structure of an acoustic radiation surface side in the prior-art ultrasonic probe;

FIG. 4 is a perspective view illustrating an appearance structure in a first ultrasonic probe in the ultrasonic probe according to the present embodiment;

FIG. 5 is a view illustrating a structure in the first ultrasonic probe on an acoustic radiation surface side;

FIG. 6 is a block diagram illustrating a control system of the ultrasonic probe according to the present embodiment;

FIG. 7 is a structural view illustrating the control system of the ultrasonic probe according to the present embodiment;

FIG. 8 is a view for explaining a calculation method of a sound speed of a shear wave when the prior-art ultrasonic probe illustrated in FIGS. 2 and 3 is used;

FIG. 9 is a diagram illustrating one example of a time waveform of the shear wave at a detection position;

FIG. 10 is a view for explaining a calculation method of a sound speed of a shear wave when the first ultrasonic probe illustrated in FIGS. 4 and 5 is used;

FIG. 11 is a diagram illustrating a structure of a head portion;

FIG. 12 is a perspective view illustrating an appearance structure in a second ultrasonic probe in the ultrasonic probe according to the present embodiment;

FIG. 13 is a view illustrating a structure of an acoustic radiation surface side in the second ultrasonic probe;

FIG. 14 is a view for explaining a calculating method of a sound speed of a shear wave when the second ultrasonic probe illustrated in FIGS. 12 and 13 is used;

FIG. 15 is a perspective view illustrating an appearance structure in a third ultrasonic probe in the ultrasonic probe according to the present embodiment;

FIG. 16 is a view illustrating a structure of an acoustic radiation surface side in the third ultrasonic probe;

FIG. 17 is a view for explaining a calculating method of a sound speed of a shear wave when the third ultrasonic probe illustrated in FIGS. 15 and 16 is used;

FIG. 18 is a perspective view illustrating an appearance structure in a fourth ultrasonic probe in the ultrasonic probe according to the present embodiment;

FIG. 19 is a view illustrating a structure of an acoustic radiation surface side in the fourth ultrasonic probe;

FIG. 20 is a view for explaining a calculating method of a sound speed of a shear wave when the fourth ultrasonic probe illustrated in FIGS. 18 and 19 is used;

FIG. 21 is a view illustrating a structure on an acoustic radiation surface side in a fifth ultrasonic probe;

FIG. 22 is a perspective view illustrating an appearance structure in a sixth ultrasonic probe in the ultrasonic probe according to the present embodiment;

FIG. 23 is a view illustrating a structure of an acoustic radiation surface side in the sixth ultrasonic probe; and

FIG. 24 is a perspective view illustrating an appearance structure in a seventh ultrasonic probe in the ultrasonic probe according to the present embodiment.

### DETAILED DESCRIPTION

An ultrasonic probe and an ultrasonic diagnosis apparatus according to a present embodiment will be described by referring to the attached drawings.

The present embodiment provides the ultrasonic probe including: at least one first transducer functioning as a transducer for excitation for executing excitation by an acoustic radiation pressure in an elastography mode; and second transducers functioning as transducers for detection for detecting a shear wave generated by the excitation in the elastography mode.

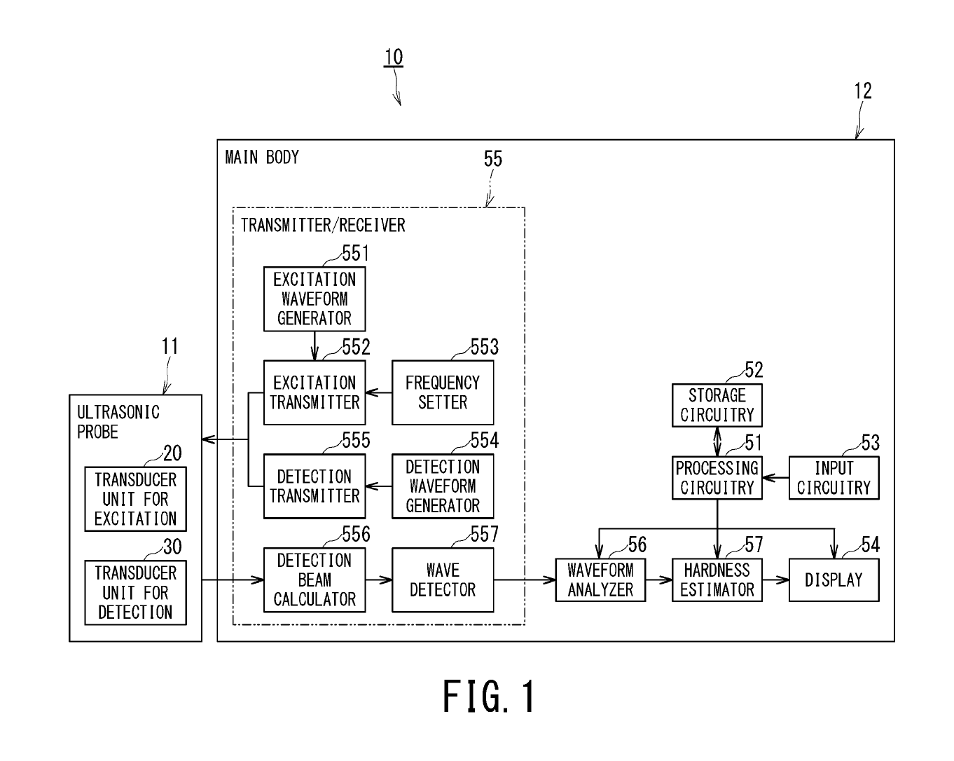

FIG. 1 is a schematic view illustrating constitution of an ultrasonic probe and an ultrasonic diagnosis apparatus according to a present embodiment.

FIG. 1 illustrates an ultrasonic diagnosis apparatus 10 according to a present embodiment. The ultrasonic diagnosis apparatus 10 includes an ultrasonic probe 11 and a main body 12.

The ultrasonic probe 11 is detachably connected to the main body 12. The ultrasonic probe 11 includes an ultrasonic transducer unit for excitation (push) in an elastography (acoustic radiation elastography) mode (hereinafter referred to as a “transducer unit 20 for excitation”) and an ultrasonic transducer unit for detection (track) of the elastography mode (hereinafter referred to as a “transducer unit 30 for detection”). The transducer unit 30 for detection is also used for transmission/reception of ultrasonic waves in a B-mode and a Doppler mode.

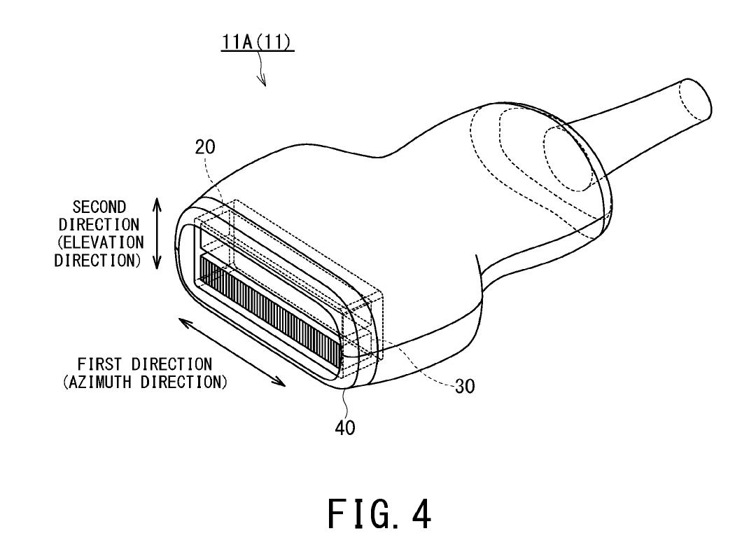

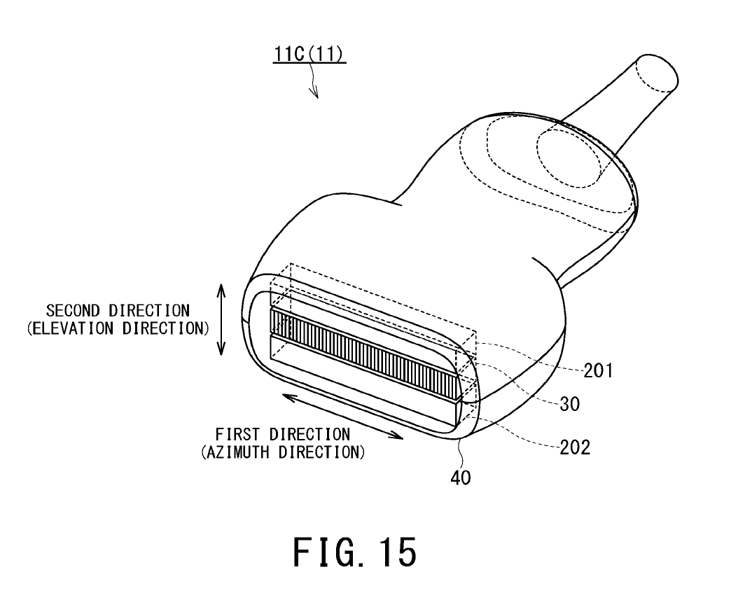

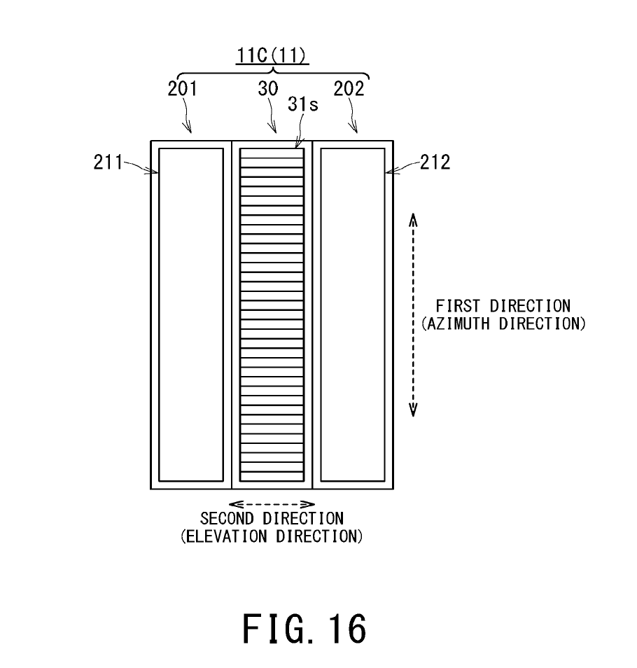

Here, a structural example when the ultrasonic probe 11 includes one transducer unit 20 for excitation is illustrated in FIGS. 4 and 5, FIGS. 12 and 13, FIGS. 18 and 19, and FIG. 21. Moreover, a structural example when the ultrasonic probe 11 includes the two transducer units 20 (201, 202) for excitation is illustrated in FIGS. 15 and 16. By defining one direction of an acoustic radiation surface of the ultrasonic probe 11 as a first direction (azimuth direction) and another direction as a second direction (elevation direction), the transducer unit 30 for detection is provided on a side of the transducer unit 20 for excitation along the second direction.

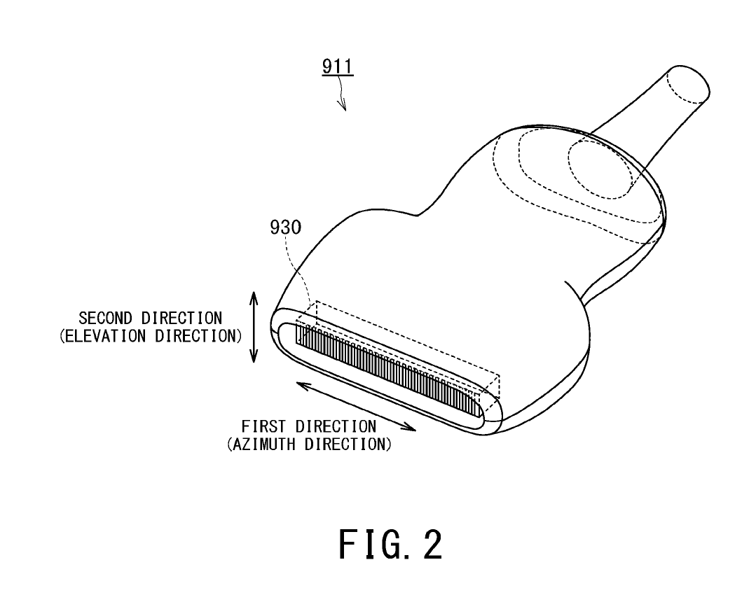

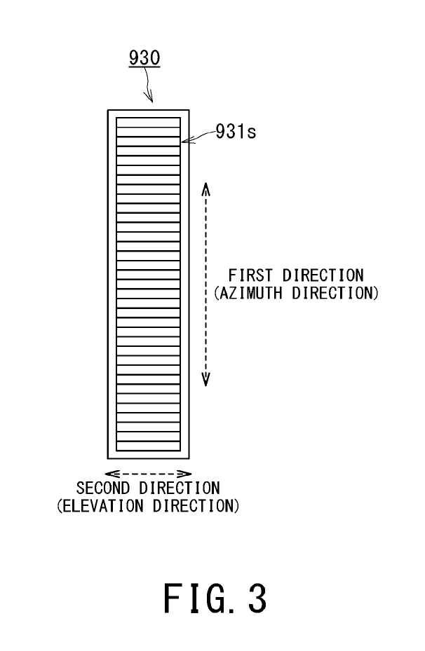

FIG. 2 is a perspective view illustrating an appearance structure in a prior-art ultrasonic probe. FIG. 3 is a view illustrating a structure of an acoustic radiation surface side in the prior-art ultrasonic probe.

FIG. 2 illustrates an appearance structure of a prior-art ultrasonic probe 911. The prior-art ultrasonic probe 911 includes one ultrasonic transducer unit used both for excitation and detection in the elastography mode (hereinafter referred to as a “transducer unit 930 both for excitation and detection”) and a cable (not illustrated) for transmitting a signal to/from the main body. The transducer unit 930 both for excitation and detection is also used for transmission/reception of ultrasonic waves in the B-mode and the Doppler mode.

As illustrated in FIG. 3, the transducer unit 930 both for excitation and detection includes multiple transducers 931s along the first direction (azimuth direction). The transducer unit 930 both for excitation and detection also includes an acoustic matching layer, a backing, an acoustic lens and the like but they are not illustrated in FIGS. 2 and 3.

Each of the multiple transducers 931s transmits ultrasonic waves for excitation with relatively large energy (sound pressure) generating an acoustic radiation pressure and also transmits/receives ultrasonic waves for detection with relative smaller energy than the ultrasonic waves for excitation.

Moreover, the multiple transducers 931s are also used in the B mode and the like other than the elastography mode. In the B mode, by sequentially switching the position of the ultrasonic beam (scanning line) for the B mode to the first direction, a still image can be also obtained. Moreover, the multiple transducers 931s can also obtain moving images by obtaining the still images in multiple frames in the B mode.

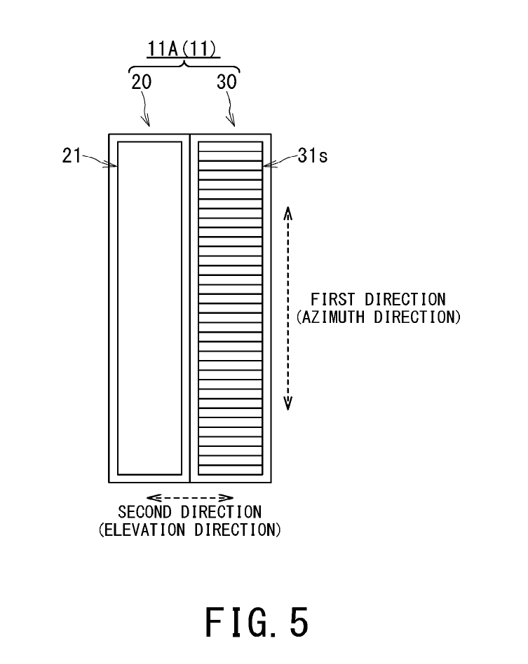

FIG. 4 is a perspective view illustrating the appearance structure in the first ultrasonic probe in the ultrasonic probe 11 according to the present embodiment. FIG. 5 is a view illustrating a structure in the first ultrasonic probe on an acoustic radiation surface side.

FIG. 4 illustrates the appearance structure of the first ultrasonic probe 11A in the ultrasonic probe 11 according to the present embodiment. The first ultrasonic probe 11A has one transducer unit 20 for excitation, one transducer unit 30 for detection, a head portion (exterior component) 40, and a cable (not illustrated) for transmitting a signal with the main body 12 (illustrated in FIG. 1) provided. The transducer unit 30 for detection is provided on one side along the second direction of the transducer unit 20 for excitation.

The transducer unit 20 for excitation includes at least one first transducer functioning as a transducer for excitation executing excitation by the acoustic radiation pressure in the elastography mode. In the example illustrated in FIG. 5, the transducer unit 20 for excitation includes one first transducer 21 with a large diameter. Hereinafter the first transducer with the large diameter is called a “large-diameter transducer”. The large-diameter transducer 21 has a width in the first direction longer that each transducer provided in the transducer unit 30 for detection, and the width in the second direction does not matter.

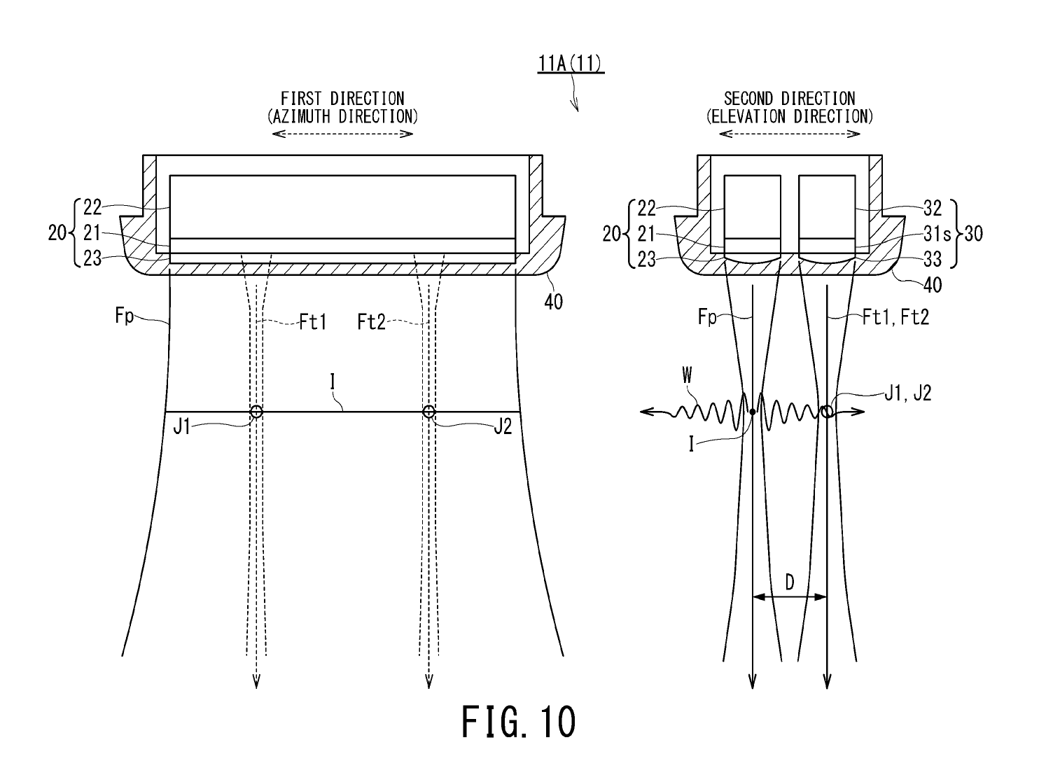

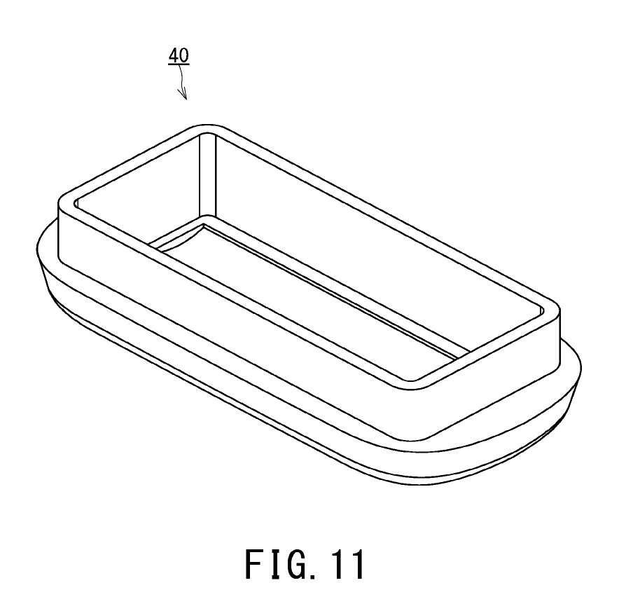

The large-diameter transducer 21 transmits the ultrasonic waves for excitation with relatively large energy generating the acoustic radiation pressure. The large-diameter transducer 21 has a certain degree of width in the first direction so that the ultrasonic waves for excitation transmitted from the large-diameter transducer 21 become a planar wave Fp (illustrated in FIG. 10) having a width in the first direction through an acoustic lens (not illustrated) focusing in the second direction. The transducer unit 20 for excitation also includes an acoustic matching layer, a backing, an acoustic lens and the like but they are not illustrated in FIGS. 4 and 5.

The transducer unit 30 for detection includes multiple second transducers functioning as transducer for detection for detecting a shear wave generated by excitation in the elastography mode. In the example illustrated in FIG. 5, the transducer unit 30 for detection includes the multiple second transducers 31s along the first direction. Each of the multiple second transducers 31s transmits/receives ultrasonic waves for detection with relatively smaller energy than the ultrasonic waves for excitation. The transducer unit 30 for detection also includes an acoustic matching layer, a backing, an acoustic lens and the like, but they are not illustrated in FIGS. 4 and 5.

Moreover, the multiple second transducers 31s are also used in the B mode and the like other than the elastography mode. In the B mode, by sequentially switching the position of the ultrasonic beam (scanning line) for the B mode to the first direction, a still image can be obtained. Moreover, the multiple second transducers 31s can also obtain moving images by obtaining the still images in multiple frames in the B mode.

Returning to the description of FIG. 1, the main body 12 includes a processing circuitry 51, a storage circuitry 52, an input circuitry 53, a display 54, a transmitter/receiver (transmission/reception circuit) 55, a waveform analyzer (waveform analysis circuit) 56, and a hardness estimator (hardness estimation circuit) 57. In the main body 12 illustrated in FIG. 1, only configuration required for executing acoustic radiation elastography is illustrated, but functions provided in a general ultrasonic diagnosis apparatus such as configuration for generating and displaying a B-mode image and a Doppler image may be also provided. Moreover, the hardness estimator 57 may be realized as a function by the processing circuitry 51 executing a program.

The processing circuitry 51 includes a CPU (central processing unit) and a memory. The processing circuitry 51 integrally controls each unit of the main body 12. The processing circuitry 51 receives an output of the transmitter/receiver 55 and can generate information indicating hardness of a tissue such as an organ in a living body and the like by controlling the waveform analyzer 56 conducting the waveform analysis and the hardness estimator 57.

The processing circuitry 51 means a processing circuitry such as an application specific integrated circuit (ASIC) and a programmable logic device in addition to an exclusive or general-purpose CPU (central processing unit) or an MPU (micro processor unit). As the programmable logic device, circuits such as a simple programmable logic device (SPLD), a complex programmable logic device (CPLD), and a field programmable gate array (FPGA) can be cited. The processing circuitry 51 reads out and executes programs stored in the storage circuitry 52 or directly incorporated in the processing circuitry 51.

Moreover, the processing circuitry 51 may be constituted by a single circuit or may be constituted by a combination of multiple independent circuits. In the latter case, multiple storage circuitries 52 storing the program may be provided individually for the respective circuits or one storage circuitry 52 may store the program corresponding to functions of the multiple circuits.

The storage circuitry 52 is a magnetic disk (hard disk and the like), an optical disk (CD-ROM, DVD and the like), a recording medium such as a semiconductor memory, and a device for reading out information stored in these mediums. In the storage circuitry 52, control programs for executing transmission/reception conditions, predetermined scanning sequences, image generation, and display processing, various signal data and image data and other data are stored. The data in the storage circuitry 52 can be transferred to an external device (not illustrated).

The input circuitry 53 is a circuit for inputting signals from various switches, buttons, a trackball, a mouse, a keyboard and the like for taking in various instructions from an operator, conditions, setting instructions of regions of interest (ROI), various image quality condition setting instructions and the like into the main body 12. Here, an input device itself is assumed to be included in the input circuitry 53. When the input device is operated by the operator, the input circuitry 53 generates an input signal according to the operation and outputs it to the processing circuitry 51. The main body 12 may include a touch panel in which the input device is constituted integrally with the display 54.

The display 54 displays an elastography image generated by the hardness estimator 57 in accordance with a control signal from the processing circuitry 51. The display 54 is a display device such as a liquid crystal display panel, a plasma display panel, an organic EL panel and the like.

The transmitter/receiver 55 controls transmission of the ultrasonic waves for excitation in the ultrasonic probe 11. The transmitter/receiver 55 includes an excitation waveform generator 551, an excitation transmitter 552, and a frequency setter 553. The excitation transmitter 552 transmits a wave transmission signal based on the waveform generated by the excitation waveform generator 551 to the transducer unit 20 for excitation under control of the processing circuitry 51.

The wave transmission signal from the excitation transmitter 552 is converted to an ultrasonic signal in the large-diameter transducer 21 (illustrated in FIG. 5) of the transducer unit 20 for excitation and transmitted. As a result, an excitation plane Fp (illustrated in FIG. 10) is formed from the transducer unit 20 for excitation toward the tissue. Transmission start time and transmission end time of the ultrasonic waves for excitation are set by the frequency setter 553. Here, the frequency means a repetition frequency of transmission of the ultrasonic waves for excitation.

Moreover, the transmitter/receiver 55 controls transmission/reception of the ultrasonic waves for detection in the ultrasonic probe 11. The transmitter/receiver 55 includes a detection waveform generator 554, a detection transmitter 555, a detection beam calculator 556, and a wave detector 557. The detection transmitter 555 transmits the ultrasonic waves for excitation under control of the processing circuitry 51 and then, transmits a wave transmission signal electronically focused (transmission delay time and/or reception delay time) in the first direction to the transducer unit 30 for detection so that detection beams Ft1 and Ft2 (illustrated in FIG. 10) based on the waveform generated by the detection waveform generator 554 are formed.

The wave transmission signal from the detection transmitter 555 is converted to the ultrasonic signal in the multiple second transducers 31s (illustrated in FIG. 5) of the transducer unit 30 for detection and transmitted. As a result, the detection beams Ft1 and Ft2 (illustrated in FIG. 10) focused by an acoustic lens 23 in the second direction are transmitted/received to/from the transducer unit 30 for detection.

Moreover, the multiple second transducers 31s of the transducer unit 30 for detection receive an echo signal caused by a shear wave W (illustrated in FIG. 10) propagating in the second direction by displacement of the tissue and convert it to an electric signal. The transducer unit 30 for detection sends the electric signal to the detection beam calculator 556. An output of the detection beam calculator 556 is subjected to signal processing such as envelope detection, log compression, band-pass filter processing, gain control and the like in the wave detector 557 and then, output as a signal indicating a change of the tissue involved in propagation of the shear wave to the waveform analyzer 56.

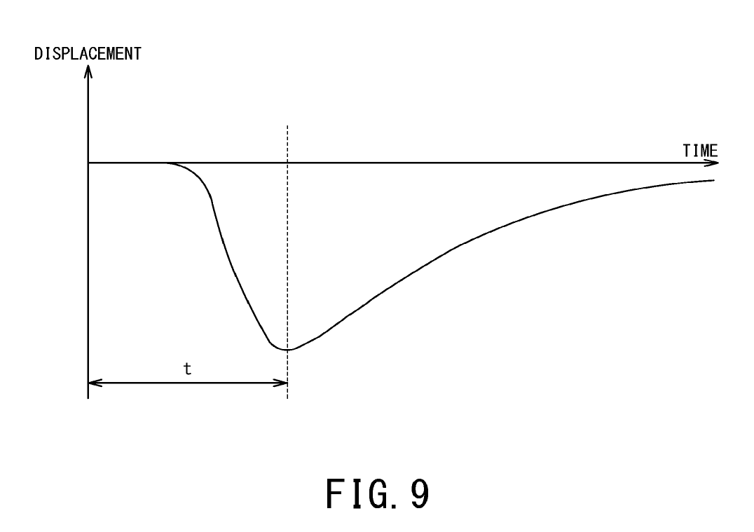

The waveform analyzer 56 makes analysis relating to the shear wave based on the signal input from the wave detector 557 of the transmitter/receiver 55. An analysis relating to the shear wave includes, for example, detection of a peak from a time waveform of the shear wave (corresponding to a graph illustrated in FIG. 9) and calculation for measuring time to have a peak (corresponding to “t” illustrated in FIG. 9). An output of the waveform analyzer 56 is output as a signal indicating a detection position and an analysis result of the shear wave to the hardness estimator 57. This analysis result is a signal indicating time to have a peak of displacement of the tissue by the shear wave, for example.

The hardness estimator 57 calculates a sound speed of the shear wave at each detection position based on the signal input from the waveform analyzer 56 and calculates an average sound speed of the shear wave on the basis of the sound speeds at the multiple detection positions. The hardness estimator 57 estimates a relative value to the average sound speed of each sound speed as hardness (modulus of elasticity) of the tissue. The hardness estimator 57 converts a signal indicating hardness of the tissue to an image signal and has a numeral value indicating the hardness of the tissue and an elastography image indicating distribution of attribution information of a color according to a degree of the numeral value indicating the hardness of the tissue (including information of at least any one of hue information, brightness information, and chroma information) displayed on the display 54.

Moreover, the hardness estimator 57 can also superpose the elastography image on a B-mode image by the B mode executed alternately with the elastography mode and display it on the display 54. Moreover, the hardness estimator 57 can also display multiple frames of elastography images on the display 54.

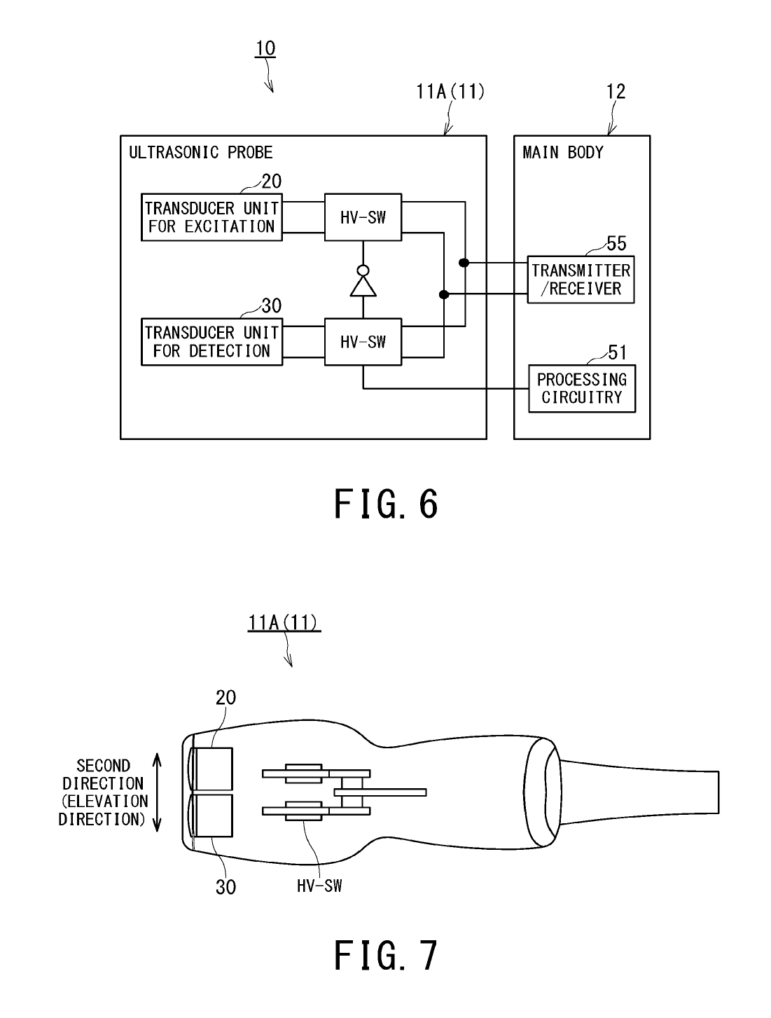

FIG. 6 is a block diagram illustrating a control system of the ultrasonic probe according to the present embodiment. FIG. 7 is a structural view illustrating the control system of the ultrasonic probe according to the present embodiment.

FIGS. 6 and 7 illustrate a first ultrasonic probe 11A of the ultrasonic diagnosis apparatus 10 and the main body 12. In order to switch timing for transmission of the ultrasonic waves for excitation and transmission/reception of the ultrasonic waves for detection, the transducer units 20 and 30 of the ultrasonic probe 11A are connected in parallel through a high-voltage switch (HV-SW) circuit. The HV-SW circuit is driven by the transmitter/receiver 55 of the main body 12. The processing circuitry 51 of the main body 12 subjects the HV-SW circuit to selective ON/OFF control. The HV-SW circuit is incorporated in a handle unit of the ultrasonic probe 11A as illustrated in FIG. 7.

Subsequently, a difference between a calculation method of a sound speed of the shear wave using the prior-art ultrasonic probe 911 (illustrated in FIGS. 2 and 3) and a calculation method of a sound speed of the shear wave using the first ultrasonic probe 11A (illustrated in FIGS. 4 and 5) will be described.

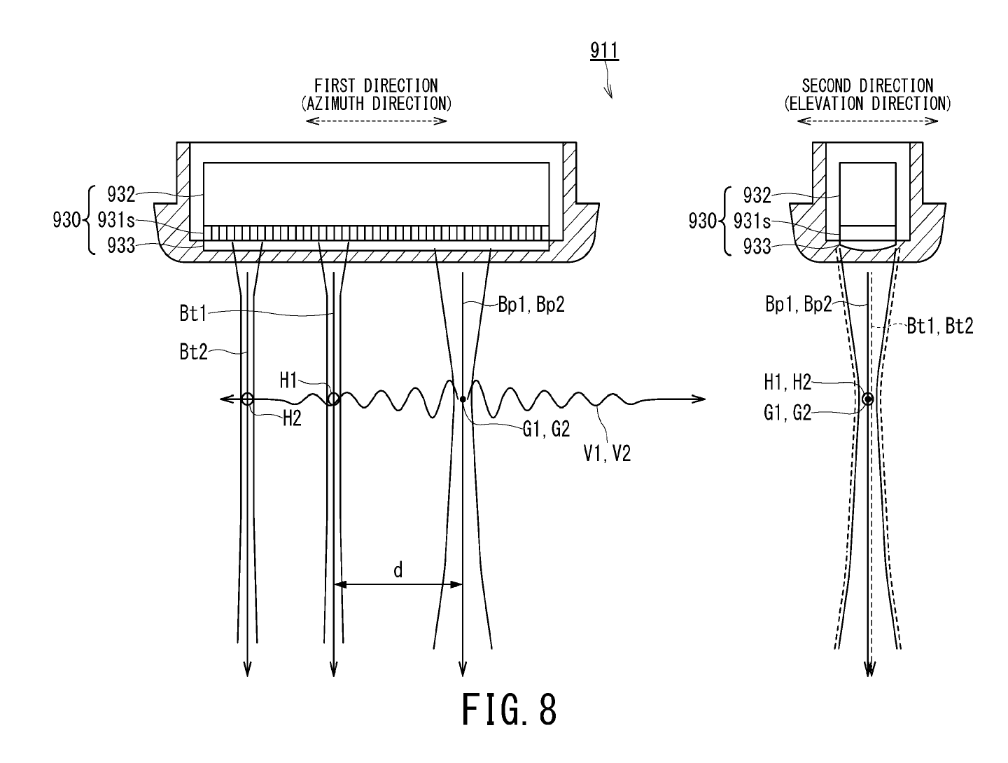

FIG. 8 is a view for explaining the calculation method of the sound speed of the shear wave when the prior-art ultrasonic probe 911 illustrated in FIGS. 2 and 3 is used.

FIG. 8 illustrates a sectional view of two orthogonal directions of the prior-art ultrasonic probe 911. The prior-art ultrasonic probe 911 has the transducer unit 930 both for excitation and detection provided. The transducer unit 930 both for excitation and detection includes multiple transducers 931s along the first direction, a backing 932, and an acoustic lens 933.

By using FIG. 8, detection of a wave crest of a shear wave by two detection points H1 and H2 along the first direction will be described.

First, the multiple transducers 931s of the transducer unit 930 both for excitation and detection transmit an ultrasonic pulse (excitation pulse) for excitation electronically focused in the first direction so as to be focused to an excitation position G1. The excitation pulse is focused to the excitation position G1 by the acoustic lens 933 focusing in the second direction. As a result, the transducer unit 930 both for excitation and detection forms an ultrasonic beam (excitation beam) Bp1 for excitation with respect to the excitation position G1. Moreover, since a series of the excitation pulses are repeatedly transmitted from the multiple transducers 931s, the transducer unit 930 both for excitation and detection repeatedly forms the excitation beam Bp1 with respect to the excitation position G1.

When the excitation beam Bp1 is repeatedly formed with respect to the excitation position G1, a shear wave is generated by displacement of the tissue present at the excitation position G1. Here, the shear wave originated in the excitation beam Bp1 and propagating in the first direction is referred to as V1.

Subsequently, after repeated formation of the excitation beam Bp1 with respect to the excitation position G1, the multiple transducers 931s of the transducer unit 930 both for excitation and detection transmits/receives the ultrasonic pulse (detection pulse) for detection electronically focused in the first direction so as to be focused to a detection position H1 set in advance (around the excitation position G1 in the first direction). The detection pulse is focused by the acoustic lens 933 focusing in the second direction to the detection position H1. As a result, the transducer unit 930 both for excitation and detection forms the ultrasonic beam (detection beam) Bt1 for detection with respect to the detection position H1. Moreover, since a series of the detection pulses are repeatedly transmitted/received to/from the multiple transducers 931s, the transducer unit 930 both for excitation and detection repeatedly forms the detection beam Bt1 with respect to the detection position H1.

When the detection beam Bt1 is repeatedly formed at the detection position H1, the shear wave V1 propagating in the first direction is detected. The electronic focusing in the first direction in order to form the detection beam Bt1 is based on the transmission delay time and/or the reception delay time.

Subsequently, after repeated formation of the detection beam Bt1 with respect to the detection position H1, the transducer unit 930 both for excitation and detection repeatedly forms an excitation beam Bp2 with respect to the excitation position G2. When the excitation beam Bp2 is repeatedly formed with respect to the excitation position G2, a shear wave is generated by displacement of the tissue present at the excitation position G2. Here, the shear wave originated in the excitation beam Bp2 and propagating in the first direction is referred to as V2.

Subsequently, after repeated formation of the excitation beam Bp2 to the excitation position G2, the transducer unit 930 both for excitation and detection repeatedly forms a detection beam Bt2 at the detection position H2. When the detection beam Bt2 is repeatedly formed to the detection position H2, the shear wave V2 propagating in the first direction is detected. The electronic focusing in the first direction for forming the detection beam Bt2 is based on the transmission delay time and/or the reception delay time.

When the wave crest of the sear wave V1 generated by the excitation beam Bp1 at the detection position H1 is detected, and the traveling time of the shear wave V1 is measured, the sound speed of the shear wave V1 at the detection position H1 is calculated from “t/d” by the tissue Doppler method or the like. Here, the term “t” is traveling time (time difference) between the transmission time of the excitation beam Bp1 to the arrival time of the wave crest of the shear wave V1 at the detection position H1. Moreover, the term “d” is a distance between the excitation position G1 to the detection position H1. One example of the time waveform of the shear wave at the detection position H1 is illustrated in FIG. 9. Moreover, after calculation of the sound speed of the shear wave V1 at the detection position H1, the sound speed of the shear wave V2 generated by the excitation beam Bp2 at the detection position H2 is also calculated similarly. Moreover, an average sound speed at the two detection positions H1 and H2 is calculated.

As described above, in the prior-art ultrasonic probe 911, the wave crests of the shear waves V1 and V2 propagating in the first direction are detected, respectively, at the two detection positions H1 and H2 along the first direction. Thus, when the traveling time of the wave crests of the shear waves at the two detection positions H1 and H2 along the first direction is to be measured, respectively, by using the prior-art ultrasonic probe 911, time for performing two sessions of a transmission sequence combining transmission of a series of excitation pulses (repeated transmission) and transmission of a series of detection pulses (repeated transmission) is required.

Then, when the traveling time of the wave crests of the shear waves at the three or more detection positions H1, H2, . . . along the first direction is to be measured, respectively, time for performing the transmission sequence in the number of sessions equal to the number of detection positions is required.

FIG. 10 is a view for explaining a calculation method of the sound speed of the shear wave when the first ultrasonic probe 11A illustrated in FIGS. 4 and 5 is used.

FIG. 10 illustrates a sectional view of two orthogonal directions of the first ultrasonic probe 11A. The first ultrasonic probe 11A includes the transducer units 20 and 30 and the head portion 40. The transducer unit 20 for excitation includes the large-diameter transducer 21, a backing 22, and the acoustic lens 23. The transducer unit 30 for detection includes multiple second transducers 31s, a backing 32, and the acoustic lens 33 along the first direction.

As materials for the acoustic lenses 23 and 33, a resin having acoustic impedance close to that of the head portion 40 and a different sound speed or silicon rubber, for example is selected in general. However, the acoustic lenses 23 and 33 may be formed by a rubber member having a shape brought into close contact with a recess portion formed in an inner surface of the head portion 40 or may be formed of an adhesive for bonding the transducer units 20 and 30 to the head portion 40.

The head portion 40 has a shape matching the shapes of the transducer units 20 and 30 in order to fix the transducer units 20 and 30 to the main body of the first ultrasonic probe 11A. The head portion 40 has a structure as illustrated in FIG. 11, and a contact surface with a body surface of a living body is smooth. As the material for the head portion 40, a resin with favorable acoustic matching with the body surface or polymethylpentene, for example, is selected.

By using FIG. 10, a case in which the wave crest of the shear wave at two detection points J1 and J2 along the first direction is detected will be described.

First, the large-diameter transducer 21 of the transducer unit 20 for excitation transmits an excitation pulse. The excitation pulse is focused by the acoustic lens 23 focusing in the second direction to an excitation region I (collection of multiple excitation positions extending in the first direction). As a result, the transducer unit 20 for excitation forms an ultrasonic plane (excitation plane) Fp for excitation to the excitation region I. Moreover, since a series of excitation pulses are repeatedly transmitted from the large-diameter transducer 21, the transducer unit 20 for excitation repeatedly forms the excitation plane Fp to the excitation region I.

When the excitation plane Fp is repeatedly formed to the excitation region I, displacement of the tissue present in the excitation region I generates a shear wave. There, the shear wave originated in the excitation plate Fp and propagating in the second direction is referred to as W.

The excitation plane Fp formed by the transducer unit 20 for excitation is focused by the acoustic lens 23 in the second direction but since it has no focusing effect in the first direction, a substantially planar-state wave surface is maintained. The linear excitation region I extending in the first direction at a certain depth is formed, and the shear wave W generated by displacement of the tissue present in the excitation region I propagates in the second direction.

Subsequently, after the repeated formation of the excitation plane Fp to the excitation region I, the multiple second transducers 31s of the transducer unit 30 for detection transmits/receives detection pulses electronically focused in the first direction so as to be focused to the detection position J1 (periphery of the excitation region I in the second direction). The detection pulse is focused to the detection position J1 by the acoustic lens 33 focusing in the second direction. As a result, the transducer unit 30 for detection forms the detection beam Ft1 to the detection position J1. Moreover, since a series of the detection pulses are repeatedly transmitted/received from the multiple second transducers 31s, the transducer unit 30 for detection repeatedly forms the detection beam Ft1 to the detection position J1.

When the detection beam Ft1 is repeatedly formed to the detection position J1, the shear wave W propagating in the second direction is detected. The electronic focusing in the first direction for forming the detection beam Ft1 is based on the transmission delay time and/or the reception delay time.

Moreover, after the repeated formation of the excitation plane Fp to the excitation region I, the transducer unit 30 for detection repeatedly forms the detection beam Ft2 to the detection position J2 in parallel with (at a same time as) the repeated formation of the detection beam Ft1 to the detection position J1. When the detection beam Ft2 is repeatedly formed to the detection position J2, the shear wave W propagating in the second direction is detected. The electronic focusing in the first direction for forming the detection beam Ft2 is based on the transmission delay time and/or the reception delay time.

When the wave crest of the shear wave W generated by the excitation plane Fp at the detection position J1 is detected and the traveling time of the shear wave W is measured, the sound speed of the shear wave W at the detection position J1 is calculated by the tissue Doppler method or the like. Moreover, in parallel with calculation of the sound speed of the shear wave W at the detection position J1, the sound speed of the shear wave W generated by the excitation plane Fp at the detection position J2 is also calculated similarly. Moreover, the average sound speed at the two detection positions J1 and J2 is calculated.

As described above, in the first ultrasonic probe 11A, the wave crests of the shear waves W propagating in the orthogonal second direction are detected at the two detection positions J1 and J2 along the first direction, respectively. Thus, when the traveling time of the wave crests of the shear waves W at the two detection positions J1 and J2 along the first direction is to be measured, respectively, by using the first ultrasonic probe 11A, transmission of the series of excitation pulses needs to be performed only one session, and detection operations at the two detection positions J1 and J2 are performed in parallel. Therefore, in the first ultrasonic probe 11A, even when the traveling time of the wave crests of the shear wave W is measured at the two detection positions J1 and J2, respectively, time only for performing one session of the transmission sequence is sufficient.

Then, in the first ultrasonic probe 11A, even when the traveling time of the wave crests of the shear waves is measured, respectively, at three or more detection positions J1, J2, . . . along the first direction, time only for performing one session of the transmission sequence is sufficient. Thus, according to the first ultrasonic probe 11A, the frame rate of the elastography image and the frame rate of the B-mode image by the B-mode performed alternately with the elastography mode are improved.

Moreover, as illustrated in FIG. 8, when the prior-art ultrasonic probe 911 is used, an interval between the excitation position G and the detection position H1 and an interval between the excitation position G and the detection position H2 are different. At the detection position H2 with a larger interval, there is a problem that the shear wave V is made dull by the propagation. In that case, since detection accuracy of the wave crest of the shear wave V deteriorates, uniformity of the image quality of the entire elastography image deteriorates. On the other hand, as illustrated in FIG. 10, when the first ultrasonic probe 11A is used, an interval (shortest distance) D between the excitation region I and the multiple detection positions J1 and J2 has a certain value. Thus, when the first ultrasonic probe 11A is used, the uniformity of the image quality of the entire elastography image is improved.

Here, the B-mode image is generated based on the ultrasonic waves for the B-mode transmitted from the multiple second transducers 31s of the transducer unit 30 for detection before or after a set of formation of the excitation plane Fp and formation of the detection beams Ft1 and Ft2.

Moreover, according to the prior-art ultrasonic probe 911 illustrated in FIG. 8, the repetition frequency of the series of excitation pulses is restricted by a frequency characteristic of the transducer unit 930 both for excitation and detection. On the other hand, according to the first ultrasonic probe 11A illustrated in FIG. 10, the transducer unit 20 for excitation for transmitting the excitation pulse independently from the transducer unit 30 for detection is provided. Thus, as the large-diameter transducer 21 provided in the transducer unit 20 for excitation, the one with an optimal frequency characteristic for effectively generating an acoustic radiation pressure and the one capable of outputting optimal acoustic sound can be selected.

In the ultrasonic probe 11, a case in which the transducer unit 30 for detection has a 1D structure provided with the multiple second transducers 31s along the first direction will be described as an example. However, the transducer unit 30 for detection may have a 2D structure provided with multiple transducers along the first direction and the second direction. In that case, the acoustic lens 33 is not needed for the transducer unit 30 for detection, and electronic focusing is performed not only in the first direction but also in the second direction.

### (Second Ultrasonic Probe)

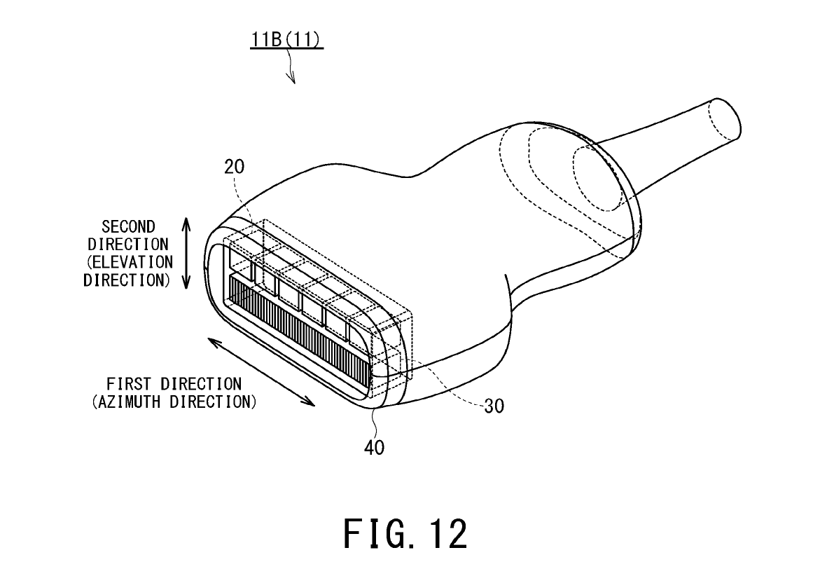

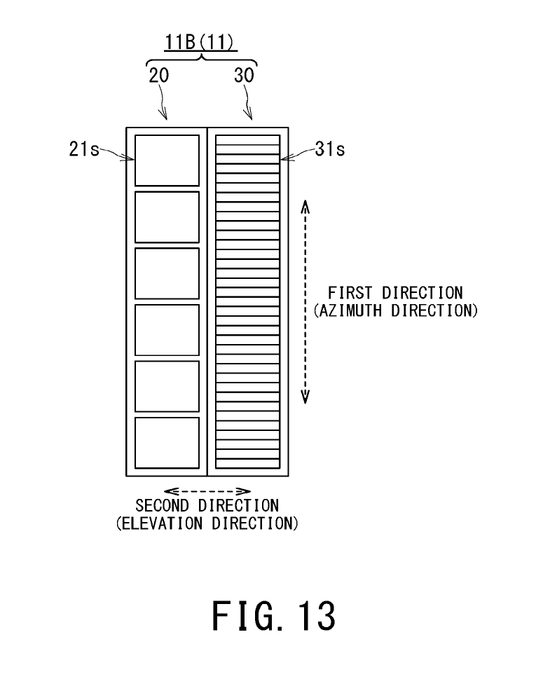

FIG. 12 is a perspective view illustrating an appearance structure in a second ultrasonic probe in the ultrasonic probe 11 according to the present embodiment. FIG. 13 is a view illustrating a structure of an acoustic radiation surface side in the second ultrasonic probe.

FIG. 12 illustrates an appearance structure of the second ultrasonic probe 11B in the ultrasonic probe 11 according to the present embodiment. The second ultrasonic probe 11B includes one transducer unit 20 for excitation, one transducer unit 30 for detection, the head portion 40, and the cable (not illustrated) for transmitting a signal with the main body 12 (illustrated in FIG. 1). The transducer unit 30 for detection is provided on one side along the second direction of the transducer unit 20 for excitation.

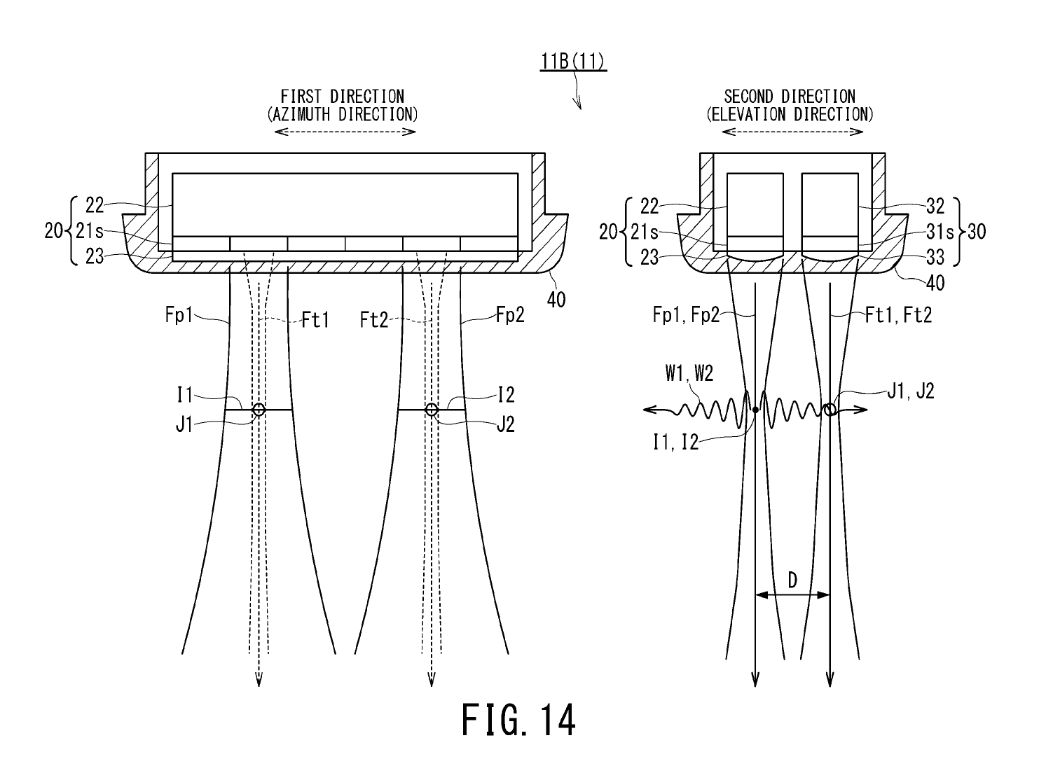

As illustrated in FIG. 13, the transducer unit 20 for excitation includes one large-diameter transducer in each region of multiple regions divided along the first direction (multiple large-diameter transducers 21s corresponding to each of the multiple regions). Each transducer of the large-diameter transducers 21s transmits ultrasonic waves for exciting relatively large energy generating an acoustic radiation pressure. Each transducer of the large-diameter transducers 21s has a certain degree of width in the first direction so that the ultrasonic waves for excitation transmitted from each transducer become planar waves Fp1 and Fp2 (illustrated in FIG. 14) each having a width in the first direction through the acoustic lens (not illustrated) focusing in the second direction. The transducer unit 20 for excitation also includes an acoustic matching layer, a backing, an acoustic lens and the like but they are not illustrated in FIGS. 12 and 13.

Since a structure and a function of the transducer unit 30 for detection illustrated in FIGS. 12 and 13 are equal to those illustrated in FIGS. 4 and 5, explanation will be omitted.

FIG. 14 is a view for explaining a calculating method of a sound speed of a shear wave when the second ultrasonic probe 11B illustrated in FIGS. 12 and 13 is used.

FIG. 14 is a sectional view of two orthogonal directions of the second ultrasonic probe 11B. The second ultrasonic probe 11B includes the transducer units 20 and 30 and the head portion 40. The transducer unit 20 for excitation includes the large-diameter transducer 21s, the backing 22, and the acoustic lens 23. The transducer unit 30 for detection includes the multiple second transducers 31s along the first direction, the backing 32, and the acoustic lens 33.

By using FIG. 14, a case in which the wave crest of the shear wave at the two detection points J1 and J2 along the first direction is detected will be described.

First, one transducer of the multiple large-diameter transducers 21s of the transducer unit 20 for excitation transmits excitation pulses. The excitation pulses are focused by the acoustic lens 23 focusing in the second direction to an excitation region I1. As a result, the transducer unit 20 for excitation forms the excitation plane Fp1 to the excitation region I1. Moreover, since a series of the excitation pulses are repeatedly transmitted from the transducer, the transducer unit 20 for excitation repeatedly forms the excitation plane Fp1 to the excitation region I1.

When the excitation plane Fp1 is repeatedly formed to the excitation region I1, a shear wave is generated by displacement of a tissue present in the excitation region I1. Here, a shear wave originated in the excitation plane Fp1 and propagating in the second direction is referred to as W1.

Moreover, in parallel with (at a same time as) repeated formation of the excitation plane Fp1 to the excitation region I1, the transducer unit 20 for excitation repeatedly forms the excitation plane Fp2 to an excitation region 12.

When the excitation plane Fp2 is repeatedly formed to the excitation region 12, the shear wave is generated by displacement of the tissue present in the excitation region 12. Here, the shear wave originated in the excitation plane Fp2 and propagating in the second direction is referred to as W2.

The excitation planes Fp1 and Fp2 formed by the transducer unit 20 for excitation are focused by the acoustic lens 23 in the second direction but since it has no focusing effect in the first direction, a substantially planar-state wave surface is maintained. The linear excitation regions I1 and I2 extending in the first direction at a certain depth are formed, and the shear waves W1 and W2 generated by displacement of the tissue present in the excitation regions I1 and 12 propagate in the second direction.

Subsequently, after the formation of the excitation plane Fp1 to the excitation region I1, the multiple second transducers 31s of the transducer unit 30 for detection transmits/receives detection pulses electronically focused in the first direction so as to be focused to the detection position J1 (periphery of the excitation region I1 in the second direction). The detection pulse is focused to the detection position J1 by the acoustic lens 33 focusing in the second direction. As a result, the transducer unit 30 for detection forms the detection beam Ft1 to the detection position J1. Moreover, since a series of the detection pulses are repeatedly transmitted/received from the multiple second transducers 31s, the transducer unit 30 for detection repeatedly forms the detection beam Ft1 to the detection position J1.

When the detection beam Ft1 is repeatedly formed to the detection position J1, the shear wave W1 propagating in the second direction is detected. The electronic focusing in the first direction for forming the detection beam Ft1 is based on the transmission delay time and/or the reception delay time.

Moreover, after the repeated formation of the excitation plane Fp2 to the excitation region 12, the transducer unit 30 for detection repeatedly forms the detection beam Ft2 to the detection position J2 in parallel with (at a same time as) the repeated formation of the detection beam Ft1 to the detection position J1. When the detection beam Ft2 is repeatedly formed to the detection position J2, the shear wave W2 propagating in the second direction is detected. The electronic focusing in the first direction for forming the detection beam Ft2 is based on the transmission delay time and/or the reception delay time.

When the wave crest of the shear wave W1 generated by the excitation plane Fp1 at the detection position J1 is detected and the traveling time of the shear wave W1 is measured, the sound speed of the shear wave W1 at the detection position J1 is calculated by the tissue Doppler method or the like. Moreover, in parallel with calculation of the sound speed of the shear wave W1 at the detection position J1, the sound speed of the shear wave W2 generated by the excitation plane Fp2 at the detection position J2 is also calculated similarly. Moreover, the average sound speed at the two detection positions J1 and J2 is calculated.

As described above, in the second ultrasonic probe 11B, the wave crests of the shear waves W1 and W2 propagating in the orthogonal second direction are detected, respectively, at the two detection positions J1 and J2 along the first direction. Thus, when the traveling time of the wave crests of the shear waves W1 and W2 at the two detection positions J1 and J2 along the first direction is to be measured, respectively, by using the second ultrasonic probe 11B, excitation operations for the two excitation regions I1 and 12 are performed in parallel, and detection operations at the two detection positions J1 and J2 are performed in parallel. Therefore, in the second ultrasonic probe 11B, even when the traveling time of the wave crests of the shear waves W1 and W2 are measured at the two detection positions J1 and J2, respectively, time only for per only one session of the transmission sequence is sufficient.

Then, in the second ultrasonic probe 11B, even when the traveling time of the wave crests of the shear waves is measured, respectively, at three or more detection positions J1, J2, . . . along the first direction, time only for performing one session of the transmission sequence is sufficient. Thus, according to the second ultrasonic probe 11B, the frame rate of the elastography image and the frame rate of the B-mode image by the B-mode performed alternately with the elastography mode are improved.

Moreover, when the second ultrasonic probe 11B is used, the interval D between the excitation region I1 and the detection position J1 and the interval D between the excitation region 12 and the detection position J2 have a certain value. Thus, when the second ultrasonic probe 11B is used, the uniformity of the image quality of the entire elastography image is improved.

Moreover, according to the second ultrasonic probe 11B, the transducer unit 20 for excitation for transmitting the excitation pulse independently from the transducer unit 30 for detection is provided. Thus, as the multiple large-diameter transducer 21s provided in the transducer unit 20 for excitation, the one with an optimal frequency characteristic for effectively generating an acoustic radiation pressure and the one capable of outputting optimal acoustic sound can be selected.

In addition, in the case of the second ultrasonic probe 11B, since a required region is selected from the multiple regions along the first direction, it becomes possible to form the excitation plane Fp1 (Fp2) in a limited range along the first direction instead of the entire range along the first direction, and wasteful energy consumption for transmission of the excitation pulse can be reduced. In that case, the processing circuitry 51 (illustrated in FIG. 1) selects a required region for transmission of the ultrasonic waves for excitation from the multiple regions of the transducer unit 20 for excitation. Then, the transducer unit 20 for excitation transmits the excitation pulse from the large-diameter transducer provided in the required region in the large-diameter transducer 21s under the control of the processing circuitry 51.

### (Third Ultrasonic Probe)

FIG. 15 is a perspective view illustrating an appearance structure in a third ultrasonic probe in the ultrasonic probe 11 according to the present embodiment. FIG. 16 is a view illustrating a structure of an acoustic radiation surface side in the third ultrasonic probe.

FIG. 15 illustrates an appearance structure of the third ultrasonic probe 11C in the ultrasonic probe 11 according to the present embodiment. The third ultrasonic probe 11C includes two transducer units 20 (201, 202) for excitation along the second direction, one transducer unit 30 for detection, the head portion 40, and the cable (not illustrated) for transmitting a signal with the main body 12 (illustrated in FIG. 1). The transducer unit 30 for detection is interposed between the transducer units 201 and 202 for excitation.

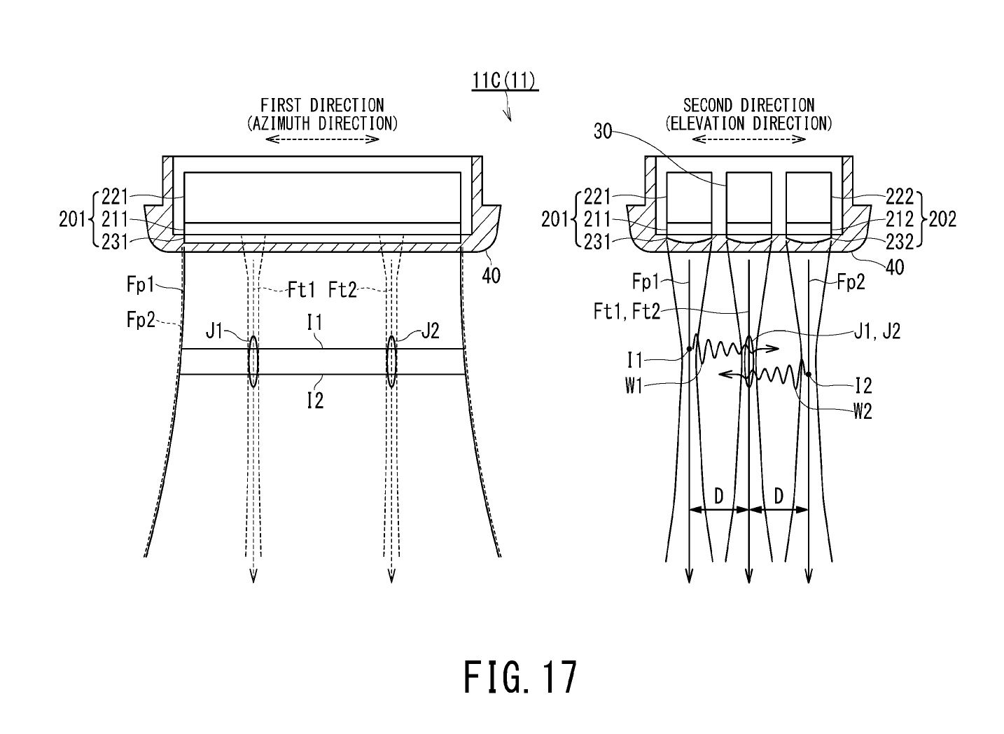

As illustrated in FIG. 16, the transducer units 201 and 202 for excitation are provided with large-diameter transducers 211 and 212, respectively. Each of the large-diameter transducers 211 and 212 transmits the ultrasonic waves for excitation with relatively large energy generating an acoustic radiation pressure. Each of the large-diameter transducers 211 and 212 has a certain degree of width in the first direction so that the ultrasonic waves for excitation transmitted from each large-diameter transducer becomes planar waves Fp1 and Fp2 (illustrated in FIG. 17) each having a width in the first direction through the acoustic lens (not illustrated) focusing in the second direction. Each of the transducer units 201 and 202 for excitation also includes an acoustic matching layer, a backing, an acoustic lens and the like but they are not illustrated in FIGS. 15 and 16.

Since a structure and a function of the transducer unit 30 for detection illustrated in FIGS. 15 and 16 are equal to those illustrated in FIGS. 4 and 5, explanation will be omitted.

FIG. 17 is a view for explaining a calculating method of a sound speed of a shear wave when the third ultrasonic probe 11C illustrated in FIGS. 15 and 16 is used.

FIG. 17 is a sectional view of two orthogonal directions of the third ultrasonic probe 11C. The third ultrasonic probe 11C includes the transducer units 201, 202, and 30 and the head portion 40. The transducer unit 201 for excitation includes the large-diameter transducer 211, the backing 221, and the acoustic lens 231. The transducer unit 202 for excitation includes the large-diameter transducer 212, the backing 222, and the acoustic lens 232. The transducer unit 30 for detection includes the multiple second transducers 31s along the first direction, the backing 32, and the acoustic lens 33.

By using FIG. 17, a case in which the wave crest of the shear wave at the two detection points J1 and J2 along the first direction will be described.

First, the large-diameter transducer 211 of the transducer unit 201 for excitation transmits excitation pulses. The excitation pulses are focused by the acoustic lens 231 focusing in the second direction to the excitation region I1. As a result, the transducer unit 201 for excitation forms the excitation plane Fp1 to the excitation region I1. Moreover, since a series of the excitation pulses are repeatedly transmitted from the large-diameter transducer 211, the transducer unit 201 for excitation repeatedly forms the excitation plane Fp1 to the excitation region I1.

When the excitation plane Fp1 is repeatedly formed to the excitation region I1, a shear wave is generated by displacement of a tissue present in the excitation region I1. Here, a shear wave originated in the excitation plane Fp1 and propagating in the second direction is referred to as W1.

Moreover, in parallel with (at a same time as) repeated formation of the excitation plane Fp1 to the excitation region I1, the transducer unit 202 for excitation repeatedly forms the excitation plane Fp2 to an excitation region 12. When the excitation plane Fp2 is repeatedly formed to the excitation region 12, the shear wave is generated by displacement of the tissue present in the excitation region 12. Here, the shear wave originated in the excitation plane Fp2 and propagating in the second direction is referred to as W2.

The excitation planes Fp1 and Fp2 formed by the transducer units 201 and 202 for excitation are focused by the acoustic lenses 231 and 232 in the second direction but since it has no focusing effect in the first direction, a substantially planar-state wave surface is maintained. The linear excitation regions I1 and 12 extending in the first direction at a certain depth are formed, and the shear waves W1 and W2 generated by displacement of the tissue present in the excitation regions I1 and 12 propagate in the second direction.

The positions of the excitation regions I1 and 12 in a depth direction are equal but in FIG. 17, they are illustrated at different positions in the depth direction for convenience.

Subsequently, after the repeated formation of the excitation planes Fp1 and Fp2 to the excitation regions I1 and I2, the multiple second transducers 31s of the transducer unit 30 for detection transmits/receives detection pulses electronically focused in the first direction so as to be focused to the detection position J1 (peripheries of the excitation regions I1 and 12 in the second direction). The detection pulse is focused to the detection position J1 by the acoustic lens 33 focusing in the second direction. As a result, the transducer unit 30 for detection forms the detection beam Ft1 to the detection position J1. Moreover, since a series of the detection pulses are repeatedly transmitted/received to/from the multiple second transducers 31s, the transducer unit 30 for detection repeatedly forms the detection beam Ft1 to the detection position J1.

When the detection beam Ft1 is repeatedly formed to the detection position J1, the shear waves W1 and W2 propagating in the second direction are detected. The electronic focusing in the first direction for forming the detection beam Ft1 is based on the transmission delay time and/or the reception delay time.

Moreover, after the repeated formation of the excitation planes Fp1 and Fp2 to the excitation regions I1 and 12, the transducer unit 30 for detection repeatedly forms the detection beam Ft2 to the detection position J2 in parallel with (at a same time as) the repeated formation of the detection beam Ft1 to the detection position J1. When the detection beam Ft2 is repeatedly formed to the detection position J2, the shear waves W1 and W2 propagating in the second direction is detected. The electronic focusing in the first direction for forming the detection beam Ft2 is based on the transmission delay time and/or the reception delay time.

When the wave crests of the shear waves W1 and W2 generated by the excitation planes Fp1 and Fp2 at the detection position J1 is detected, respectively, and the traveling time (average value) of the shear waves W1 and W2 is measured, the sound speed (average value) of the shear waves W1 and W2 at the detection position J1 is calculated by the tissue Doppler method or the like. Moreover, in parallel with calculation of the sound speeds of the shear waves W1 and W2 at the detection position J1, the sound speed of the shear waves W1 and W2 generated by the excitation planes Fp1 and Fp2 at the detection position J2 is also calculated similarly. Moreover, the average sound speed at the two detection positions J1 and J2 is calculated.

As described above, in the third ultrasonic probe 11C, the wave crests of the shear waves W1 and W2 propagating in the orthogonal second direction are detected at the two detection positions J1 and J2 along the first direction. Thus, when the traveling time of the wave crests of the shear waves W1 and W2 at each of the two detection positions J1 and J2 along the first direction is to be measured, respectively, by using the third ultrasonic probe 11C, excitation operations for the two excitation regions I1 and 12 are performed in parallel, and detection operations at the two detection positions J1 and J2 are performed in parallel. Therefore, in the third ultrasonic probe 11C, even when the traveling time of the wave crests of the shear waves W1 and W2 is measured at the two detection positions J1 and J2, respectively, time only for performing one session of the transmission sequence is sufficient.

Then, in the third ultrasonic probe 11C, even when the traveling time of the wave crests of the shear waves is measured, respectively, at three or more detection positions J1, J2, . . . along the first direction, time only for performing one session of the transmission sequence is sufficient. Thus, according to the third ultrasonic probe 11C, the frame rate of the elastography image and the frame rate of the B-mode image by the B-mode performed alternately with the elastography mode are improved.

Moreover, when the third ultrasonic probe 11C is used, the interval D between the excitation region I1 and the detection position J1, the interval D between the excitation region I1 and the detection position J2, the interval D between the excitation region 12 and the detection position J1, and the interval D between the excitation region 12 and the detection position J2 have a certain value. Thus, when the third ultrasonic probe 11C is used, the uniformity of the image quality of the elastography image is improved.

Moreover, according to the third ultrasonic probe 11C, the transducer units 201 and 202 for excitation for transmitting the excitation pulse independently from the transducer unit 30 for detection is provided. Thus, as the large-diameter transducers 211 and 212 provided in the transducer units 201 and 202 for excitation, the one with an optimal frequency characteristic for effectively generating an acoustic radiation pressure can be selected.

In addition, if the elastography image is displayed by being superposed on an ordinary B-mode image obtained by using the transducer unit 30 for detection, a section of the B-mode image and a section of the elastography image are slightly different in the first ultrasonic probe 11A (illustrated in FIGS. 4 and 5) and the second ultrasonic probe 11B (illustrated in FIGS. 12 and 13). However, in the third ultrasonic probe 11C, since the transducer units 201 and 202 for excitation are arranged on both sides along the second direction of the transducer unit 30 for detection, a center axis of the transducer unit 30 for detection can be made to match the center of the section of the elastography image.

A structure of the second ultrasonic probe 11B may be combined with the third ultrasonic probe 11C. That is, each of the transducer units 201 and 202 for excitation of the third ultrasonic probe 11C may include one large-diameter transducer in each region of the multiple regions divided along the first direction (multiple large-diameter transducers corresponding to multiple regions, respectively).

### (Fourth Ultrasonic Probe)

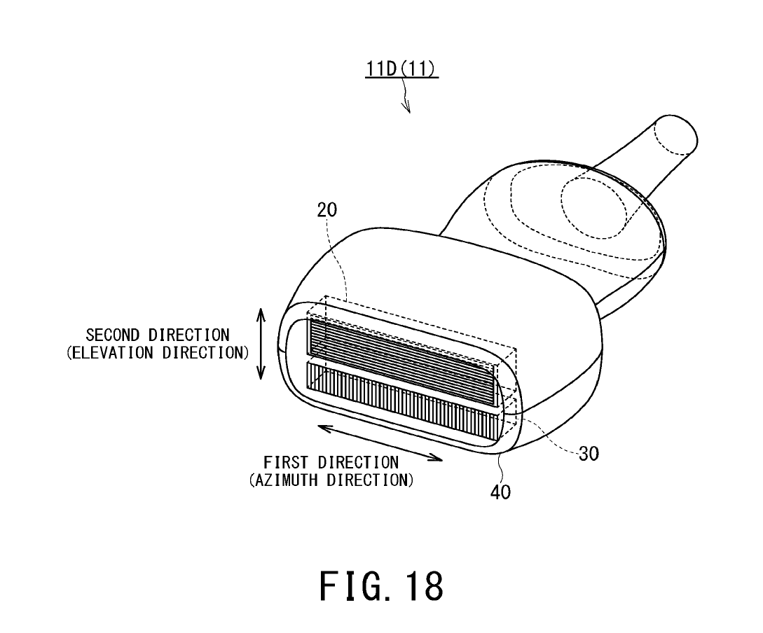

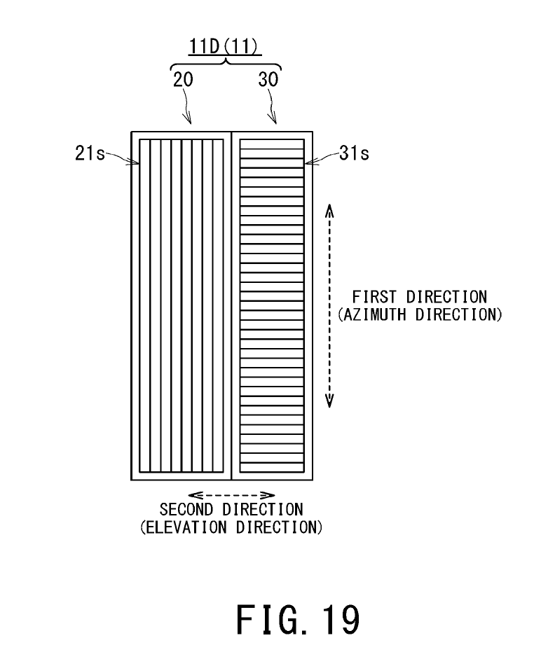

FIG. 18 is a perspective view illustrating an appearance structure in a fourth ultrasonic probe in the ultrasonic probe 11 according to the present embodiment. FIG. 19 is a view illustrating a structure of an acoustic radiation surface side in the fourth ultrasonic probe.

FIG. 18 illustrates an appearance structure of the fourth ultrasonic probe 11D in the ultrasonic probe 11 according to the present embodiment. The fourth ultrasonic probe 11D includes one transducer unit 20 for excitation, one transducer unit 30 for detection, the head portion 40, and the cable (not illustrated) for transmitting a signal with the main body 12 (illustrated in FIG. 1). The transducer unit 30 for detection is provided on one side along the second direction of the transducer unit 20 for excitation.

As illustrated in FIG. 19, a width of the transducer unit 20 for excitation in the second direction is larger than a width of the transducer unit 30 for detection in the second direction. Moreover, the transducer unit 20 for excitation includes multiple first transducers 21s along the second direction. Each transducer of the multiple first transducers 21s illustrated in FIG. 19 transmits ultrasonic waves for excitation with relatively large energy generating an acoustic radiation pressure. Though the transducer unit 20 for excitation also includes an acoustic matching layer, a backing and the like, they are not illustrated in FIGS. 15 and 16.

Since a structure and a function of the transducer unit 30 for detection illustrated in FIGS. 18 and 19 are equal to those illustrated in FIGS. 4 and 5, explanation will be omitted.

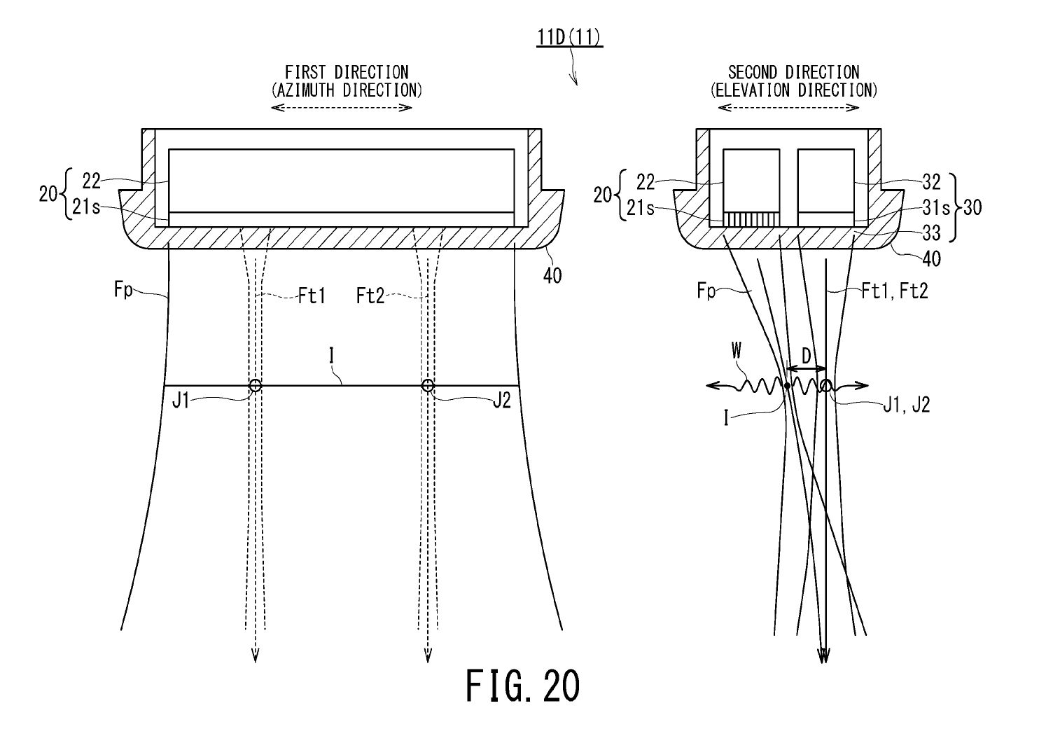

FIG. 20 is a view for explaining a calculating method of a sound speed of a shear wave when the fourth ultrasonic probe 11D illustrated in FIGS. 18 and 19 is used.

FIG. 20 is a sectional view of two orthogonal directions of the fourth ultrasonic probe 11D. The fourth ultrasonic probe 11D includes the transducer units 20 and 30 and the head portion 40. The transducer unit 20 for excitation includes the multiple first transducers 21s and the backing 22 along the second direction, and the acoustic lens does not have to be provided. The transducer unit 30 for detection includes the multiple second transducers 31s along the first direction, the backing 32, and the acoustic lens 33.

By using FIG. 20, a case in which the wave crest of the shear wave at the two detection points J1 and J2 along the first direction is detected will be described.

First, the multiple first transducers 21s of the transducer unit 20 for excitation transmit the excitation plane Fp electronically focused in the second direction so as to be focused to the excitation region I. As a result, the transducer unit 20 for excitation forms the excitation plane Fp to the excitation region I. Moreover, since a series of the excitation pulses are repeatedly transmitted from the multiple first transducers 21s, the transducer unit 20 for excitation repeatedly forms the excitation plane Fp to the excitation region I.

When the excitation plane Fp is repeatedly formed to the excitation region I, a shear wave is generated by displacement of a tissue present in the excitation region I. Here, a shear wave originated in the excitation plane Fp and propagating in the second direction is referred to as W.

The excitation plane Fp formed by the transducer unit 20 for excitation is electronically focused in the second direction but since it has no focusing effect in the first direction, a substantially planar-state wave surface is maintained. The linear excitation region I extending in the first direction at a certain depth is formed, and the shear wave W generated by displacement of the tissue present in the excitation region I propagates in the second direction.

Subsequently, after the repeated formation of the excitation plane Fp to the excitation region I, the multiple second transducers 31s of the transducer unit 30 for detection transmits/receives detection pulses electronically focused in the first direction so as to be focused to the detection position J1 (periphery of the excitation region I in the second direction). The detection pulse is focused to the detection position J1 by the acoustic lens 33 focusing in the second direction. As a result, the transducer unit 30 for detection forms the detection beam Ft1 to the detection position J1. Moreover, since a series of the detection pulses are repeatedly transmitted/received to/from the multiple second transducers 31s, the transducer unit 30 for detection repeatedly forms the detection beam Ft1 to the detection position J1.

When the detection beam Ft1 is repeatedly formed to the detection position J1, the shear wave W propagating in the second direction is detected. The electronic focusing in the first direction for forming the detection beam Ft1 is based on the transmission delay time and/or the reception delay time.

Moreover, after the repeated formation of the excitation plane Fp to the excitation region I, the transducer unit 30 for detection repeatedly forms the detection beam Ft2 to the detection position J2 in parallel with (at a same time as) the repeated formation of the detection beam Ft1 to the detection position J1. When the detection beam Ft2 is repeatedly formed to the detection position J2, the shear wave W propagating in the second direction is detected. The electronic focusing in the first direction for forming the detection beam Ft2 is based on the transmission delay time and/or the reception delay time.

When the wave crest of the shear wave W generated by the excitation plane Fp at the detection position J1 is detected and the traveling time of the shear wave W is measured, the sound speed of the shear wave W at the detection position J1 is calculated by the tissue Doppler method or the like. Moreover, in parallel with calculation of the sound speed of the shear wave W at the detection position J1, the sound speed of the shear wave W generated by the excitation plane Fp at the detection position J2 is also calculated similarly. Moreover, the average sound speed at the two detection positions J1 and J2 is calculated.

As described above, in the fourth ultrasonic probe 11D, the wave crests of the shear waves W propagating in the orthogonal second direction are detected at the two detection positions J1 and J2 along the first direction, respectively. Thus, when the traveling time of the wave crests of the shear waves W at the two detection positions J1 and J2 along the first direction is measured, respectively, by using the fourth ultrasonic probe 11D, transmission of the series of excitation pulses needs to be performed only one session, and detection operations at the two detection positions J1 and J2 are performed in parallel. Therefore, in the fourth ultrasonic probe 11D, even when the traveling time of the wave crests of the shear wave W is measured at the two detection positions J1 and J2, respectively, time only for performing one session of the transmission sequence is sufficient.

Then, in the fourth ultrasonic probe 11D, even when the traveling time of the wave crests of the shear waves is measured, respectively, at three or more detection positions J1, J2, . . . along the first direction, time only for performing one session of the transmission sequence is sufficient. Thus, according to the fourth ultrasonic probe 11D, the frame rate of the elastography image and the frame rate of the B-mode image by the B-mode performed alternately with the elastography mode are improved.

Moreover, when the fourth ultrasonic probe 11D is used, the interval D between the excitation region I and the multiple detection positions J1 and J2 has a certain value. Thus, when the fourth ultrasonic probe 11D is used, the uniformity of the image quality of the elastography image is improved.

Moreover, according to the fourth ultrasonic probe 11D, the transducer unit 20 for excitation for transmitting the excitation pulse independently from the transducer unit 30 for detection is provided. Thus, as the multiple first transducer 21s provided in the transducer unit 20 for excitation, the one with an optimal frequency characteristic for effectively generating an acoustic radiation pressure and the one capable of outputting optimal acoustic sound can be selected.

In addition, when the fourth ultrasonic probe 11D is used, electronic focusing is performed in the second direction so that the excitation plane Fp is focused to the desired excitation region I (transmission delay time is given). When the fourth ultrasonic probe 11D is used, the excitation plane Fp can be formed with a larger diameter as compared with use of the first ultrasonic probe 11A (illustrated in FIGS. 4 and 5). Moreover, in the first ultrasonic probe 11A, the excitation plane Fp is formed in accordance with a sound field determined by the acoustic lens 23 (illustrated in FIG. 10) in a fixed manner, but in the fourth ultrasonic probe 11D, an optimal sound field with respect to a depth at which the elastography image is to be obtained can be formed by controlling the electronic focusing in the second direction.

### (Fifth Ultrasonic Probe)

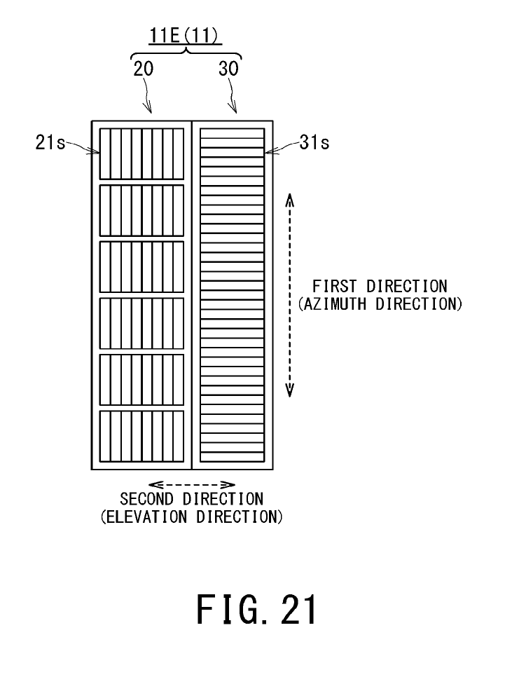

FIG. 21 is a view illustrating a structure on an acoustic radiation surface side in a fifth ultrasonic probe.

FIG. 21 illustrates a fifth ultrasonic probe 11E having a structure combining the second ultrasonic probe 11B illustrated in FIGS. 12 and 13 and a structure of the fourth ultrasonic probe 11D illustrated in FIGS. 18 and 19.

As illustrated in FIG. 21, the transducer unit 20 for excitation includes multiple first transducers 21s along the second direction in each region of multiple regions divided along the first direction. Each transducer of the multiple first transducers 21s illustrated in FIG. 21 transmits ultrasonic waves for excitation with relatively large energy generating an acoustic radiation pressure.

In the case of the fifth ultrasonic probe 11E, as described by using the second ultrasonic probe 11B in FIG. 14, the excitation plane Fp can be formed in a limited range along the first direction, and wasteful energy consumption for transmission of the excitation pulse can be reduced. In that case, the processing circuitry 51 (illustrated in FIG. 1) selects a required region for transmission of the excitation pulse in the multiple regions of the transducer unit 20 for excitation. Then, the transducer unit 20 for excitation transmits the excitation pulse from the multiple first transducers 21s provided in the required region under control of the processing circuitry 51.

Moreover, in the case of the fifth ultrasonic probe 11E, the effect similar to the case of the fourth ultrasonic probe 11D illustrated in FIG. 20 can be obtained.

### (Sixth Ultrasonic Probe)

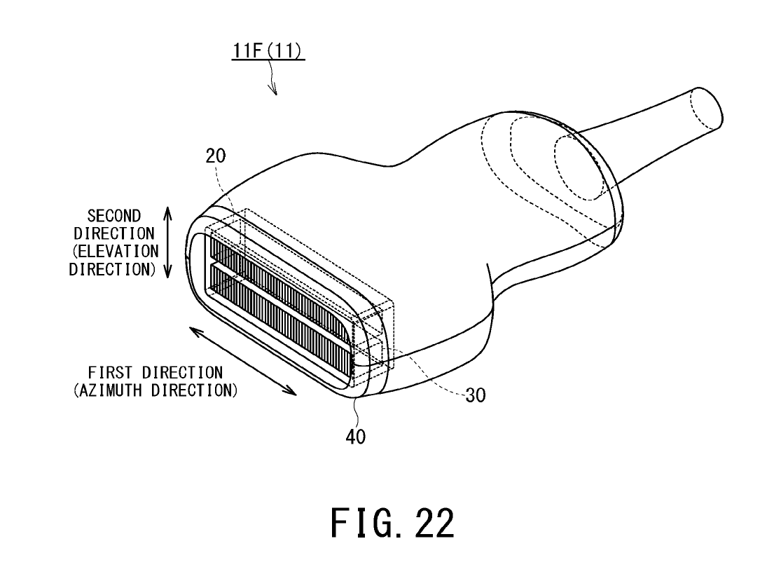

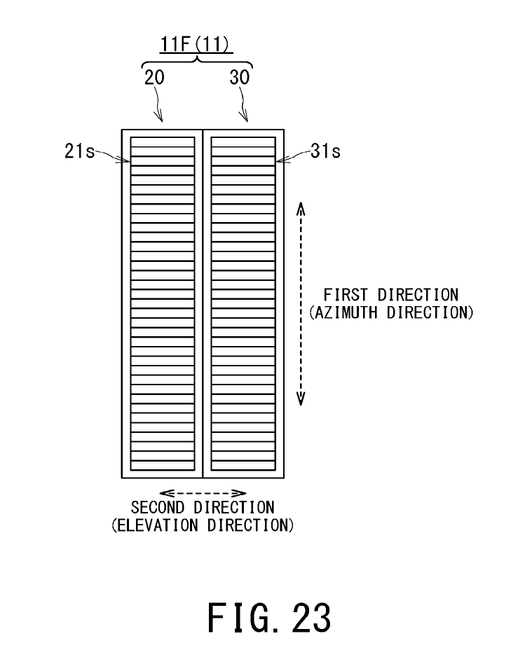

FIG. 22 is a perspective view illustrating an appearance structure in a sixth ultrasonic probe in the ultrasonic probe 11 according to the present embodiment. FIG. 23 is a view illustrating a structure of an acoustic radiation surface side in the sixth ultrasonic probe.

FIG. 22 illustrates an appearance structure of the sixth ultrasonic probe 11F in the ultrasonic probe 11 according to the present embodiment. The sixth ultrasonic probe 11F includes one transducer unit 20 for excitation, one transducer unit 30 for detection, the head portion (exterior component) 40, and the cable (not illustrated) for transmitting a signal with the main body 12 (illustrated in FIG. 1). The transducer unit 30 for detection is provided on one side along the second direction of the transducer unit 20 for excitation.

As illustrated in FIG. 23, the transducer unit 20 for excitation includes multiple first transducers 21s along the first direction. Each of the multiple first transducers 21s transmits ultrasonic waves for excitation with relatively large energy generating an acoustic radiation pressure. The multiple first transducers 21s have a certain degree of width in the first direction so that the ultrasonic waves for excitation transmitted from the multiple first transducers 21s become the planar wave Fp (illustrated in FIG. 10) having a width in the first direction through an acoustic lens (not illustrated) focusing in the second direction. The transducer unit 20 for excitation also includes an acoustic matching layer, a backing, an acoustic lens and the like but they are not illustrated in FIGS. 22 and 23.

Since a structure and a function of the transducer unit 30 for detection illustrated in FIGS. 22 and 23 are equal to those illustrated in FIGS. 4 and 5, explanation will be omitted.

By transmitting the ultrasonic waves for excitation from all the multiple first transducers 21s, the planar wave Fp is formed similarly to the case of the first ultrasonic probe 11A illustrated in FIG. 10, and the sound speed of the shear wave according to the planar wave Fp is calculated. Moreover, by transmitting the ultrasonic waves for excitation from a part of the multiple first transducers 21s, a planar wave with a width limited more than the planar wave Fp in the case of the first ultrasonic probe 11A illustrated in FIG. 10 is formed, and the sound speed of the shear wave according to this planar wave is calculated.

In the case of the sixth ultrasonic probe 11F, the excitation plane can be formed in a limited range along the first direction, and wasteful energy consumption for transmission of the excitation pulse can be reduced. In that case, the transducer unit 20 for excitation transmits the excitation pulse from a part of the multiple first transducers 21s under control of the processing circuitry 51.

Moreover, in the case of the sixth ultrasonic probe 11F, the effect similar to the case of the first ultrasonic probe 11A illustrated in FIG. 10 can be obtained. In that case, the transducer unit 20 for excitation transmits the excitation pulse from all of the multiple first transducers 21s under control of the processing circuitry 51.

### (Seventh Ultrasonic Probe)

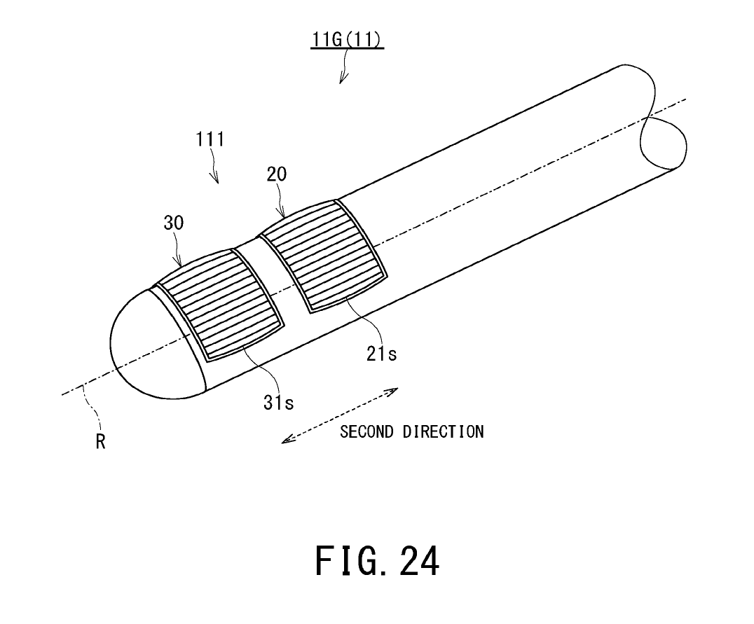

FIG. 24 is a perspective view illustrating an appearance structure in a seventh ultrasonic probe in the ultrasonic probe 11 according to the present embodiment.

FIG. 24 illustrates an appearance structure of a seventh ultrasonic probe 11G in the ultrasonic probe 11 according to the present embodiment. While the aforementioned first to sixth ultrasonic probes are external ultrasonic probes, the seventh ultrasonic probe 11G is an internal ultrasonic probe. The seventh ultrasonic probe 11G has a structure in which the structure of the sixth ultrasonic probe 11F illustrated in FIG. 22 is applied to the external ultrasonic probe, but it may have a structure in which the structures of the first to fifth ultrasonic probes 11A to 11E are applied to the external ultrasonic probe.

The seventh ultrasonic probe 11G includes an insertion portion 111 which can be inserted into an object. The insertion portion 111 includes one transducer unit 20 for excitation along the second direction and one transducer unit 30 for detection. The transducer unit 30 for detection is provided on one side along the second direction of the transducer unit 20 for excitation. The second direction follows an axis R of the sixth ultrasonic probe 11F.

As illustrated in FIG. 24, the transducer unit 20 for excitation includes multiple first transducers 21s along a third direction (circumferential direction) around the axis R of the sixth ultrasonic probe 11F. The multiple first transducers 21s are convex array.

Each of the multiple first transducers 21s transmits ultrasonic waves for excitation with relatively large energy generating an acoustic radiation pressure. The transducer unit 20 for excitation includes an acoustic matching layer, a backing, an acoustic lens and the like but they are not illustrated in FIG. 24.

On the other hand, the transducer unit 30 for detection includes multiple second transducers 31s along the third direction. The multiple second transducers 31s are convex arrays. An example in which the transducer unit 30 for detection is provided on a tip end side rather than the transducer unit 20 for excitation is illustrated but this is not limiting.

Each of the multiple second transducers 31s transmits/receives the ultrasonic waves for detection with relatively smaller energy than the ultrasonic waves for excitation. The transducer unit 30 for detection includes an acoustic matching layer, a backing, an acoustic lens and the like but they are not illustrated in FIG. 24.

Moreover, the multiple second transducers 31s are also used in the B mode and the like other than in the elastography mode. In the B mode, a still image can be obtained by sequentially switching a position of an ultrasonic beam (scanning line) for the B mode to the third direction. Moreover, the multiple second transducers 31s can obtain moving images by obtaining still images in multiple frames in the B mode.

By transmitting the ultrasonic waves for excitation from all of the multiple first transducers 21s, the planar wave Fp is formed similarly to the case of the first ultrasonic probe 11A illustrated in FIG. 10, and a sound speed of a shear wave according to the planar wave Fp is calculated. Moreover, by transmitting the ultrasonic waves for excitation from a part of the multiple first transducers 21s, a planar wave having a width limited more than the planar wave Fp in the case of the first ultrasonic probe 11A illustrated in FIG. 10 is formed, and the sound speed of the shear wave according to this planar wave is calculated.

In the case of the seventh ultrasonic probe 11G, an excitation plane can be formed in a limited range along the first direction, and wasteful energy consumption for transmission of the excitation pulse can be reduced. In that case, the transducer unit 20 for excitation transmits the excitation pulse from a part of the multiple first transducers 21s under control of the processing circuitry 51.

Moreover, in the case of the seventh ultrasonic probe 11G, the effect similar to the case of the first ultrasonic probe 11A illustrated in FIG. 10 can be obtained. In that case, the transducer unit 20 for excitation transmits the excitation pulse from all of the multiple first transducers 21s under control of the processing circuitry 51.

According to the ultrasonic probe of at least one of the aforementioned embodiments, information to generate an elastography image can be generated in time required for the minimum number of times of transmission sequences. According to the ultrasonic diagnosis apparatus of at least one of the aforementioned embodiments, an elastography image can be generated in time required for the minimum number of times of transmission sequences, and the elastography image with a high frame rate can be obtained while uniformity of the image quality of the entire elastography image is improved.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic probe comprising:
at least one first transducer functioning as a transducer for excitation for executing excitation by an acoustic radiation pressure in an elastography mode; and
second transducers functioning as transducers for detection for detecting a shear wave generated by the excitation in the elastography mode.

2. An ultrasonic probe comprising:
at least one first transducer for excitation for executing excitation by an acoustic radiation pressure to an object; and
second transducers for detecting a shear wave generated by the excitation through transmission/reception of ultrasonic waves to the object.

3. An ultrasonic probe, comprising:
at least one first transducer for executing excitation by an acoustic radiation pressure to an object; and
second transducers each having a size different from that of the first transducer and detecting a shear wave generated by the excitation through transmission/reception of ultrasonic waves.

4. The ultrasonic probe according to claim 1, further comprising:
an insertion portion to be inserted into the object, the insertion portion including the at least one first transducer and the second transducers.

5. The ultrasonic probe according to claim 1, wherein
the second transducers are arranged at least along an azimuth direction; and
the at least one first transducer comprises first transducers, and the first transducers and the second transducers are arranged in a juxtaposed manner in an elevation direction orthogonal to the azimuth direction.

6. The ultrasonic probe according to claim 1, further comprising:
an ultrasonic transducer unit for excitation including the at least one first transducer and having a width along an azimuth direction and focusing ultrasonic waves for the excitation along an elevation direction orthogonal to the azimuth direction; and
an ultrasonic transducer unit for detection including the second transducers and provided on a side along the elevation direction of the ultrasonic transducer unit for excitation.

7. The ultrasonic probe according to claim 6, wherein
one ultrasonic transducer unit for excitation as the ultrasonic transducer unit for excitation has one first transducer and an acoustic lens for focusing, along the elevation direction, the ultrasonic waves for the excitation transmitted from the one first transducer; and
the ultrasonic transducer unit for detection is provided on one side along the elevation direction of the one ultrasonic transducer unit for excitation.

8. The ultrasonic probe according to claim 6, wherein
one ultrasonic transducer unit for excitation as the ultrasonic transducer unit for excitation has one first transducer arranged in each region of multiple regions divided along the azimuth direction and an acoustic lens for focusing, along the elevation direction, ultrasonic waves for the excitation transmitted from the one first transducer in the each region; and
the ultrasonic transducer unit for detection is provided on one side along the elevation direction of the one ultrasonic transducer unit for excitation.

9. The ultrasonic probe according to claim 6, wherein
each of two ultrasonic transducer units for excitation along the elevation direction as the ultrasonic transducer units for excitation has one transducer and an acoustic lens for focusing, along the elevation direction, the ultrasonic waves for the excitation transmitted from the one transducer; and
the ultrasonic transducer unit for detection is interposed between the two ultrasonic transducer units for excitation.

10. The ultrasonic probe according to claim 6, wherein
one ultrasonic transducer unit for excitation as the ultrasonic transducer unit for excitation includes first transducers along the elevation direction; and
the ultrasonic transducer unit for detection is provided on one side along the elevation direction of the one ultrasonic transducer unit for excitation.

11. The ultrasonic probe according to claim 6, wherein
one ultrasonic transducer unit for excitation as the ultrasonic transducer unit for excitation includes first transducers along the elevation direction in each region of multiple regions divided along the azimuth direction; and
the ultrasonic transducer unit for detection is provided on one side along the elevation direction of the one ultrasonic transducer unit for excitation.

12. The ultrasonic probe according to claim 6, wherein
in order to switch timing of transmission of the ultrasonic waves for the excitation and transmission/reception of ultrasonic waves for the detection, the ultrasonic transducer unit for excitation and the ultrasonic transducer unit for detection are connected in parallel through a high-voltage switch selectively executing ON/OFF control.

13. An ultrasonic diagnosis apparatus comprising:
an ultrasonic probe according to claim 1; and
a processing circuitry for controlling transmission of ultrasonic waves for excitation in the at least one first transducer and for controlling transmission/reception of ultrasonic waves for detection in the second transducers, and
for executing control to calculate a sound speed of a shear wave based on a reception signal according to the ultrasonic waves for detection and to estimate hardness of a tissue present in the excitation region based on the sound speed.

14. The ultrasonic diagnosis apparatus according to claim 13, wherein
the ultrasonic prove has:
an ultrasonic transducer unit for excitation including the at least one first transducer, having a width along the azimuth direction, and focusing the ultrasonic waves for excitation along an elevation direction orthogonal to the azimuth direction; and
an ultrasonic transducer unit for detection including the second transducers and provided on a side along the elevation direction of the ultrasonic transducer unit for excitation.

15. The ultrasonic diagnosis apparatus according to claim 14, wherein
one ultrasonic transducer unit for excitation as the ultrasonic transducer unit for excitation has one first transducer arranged in each region of multiple regions divided along the azimuth direction and an acoustic lens focusing the ultrasonic waves for excitation transmitted from the one first transducer in the each region along the elevation direction; and
the ultrasonic transducer unit for detection is provided on one side along the elevation direction of the one ultrasonic transducer unit for excitation.

16. The ultrasonic diagnosis apparatus according to claim 15, wherein
the processing circuitry controls to select a required region for transmission of the ultrasonic waves for excitation in the multiple regions of the ultrasonic transducer unit for excitation; and
the ultrasonic transducer unit for excitation transmits the ultrasonic waves for excitation from one first transducer provided in the required region.

17. The ultrasonic diagnosis apparatus according to claim 14, wherein
the one ultrasonic transducer unit for excitation includes first transducers along the elevation direction in each region of multiple regions divided along the azimuth direction; and
the ultrasonic transducer unit for detection is provided on one side along the elevation direction of the one ultrasonic transducer unit for excitation.

18. The ultrasonic diagnosis apparatus according to claim 17, wherein
the processing circuitry controls to select a required region for transmission of the ultrasonic waves for excitation in the multiple regions of the ultrasonic transducer unit for excitation; and
the ultrasonic transducer unit for excitation transmits the ultrasonic waves for excitation from first transducers provided in the required region.

19. The ultrasonic diagnosis apparatus according to claim 14, wherein
the processing circuitry controls the ultrasonic probe to focus the ultrasonic waves for detection in parallel with detecting positions along the azimuth direction, respectively; and
controls to calculate sound speeds of the shear waves at the detection positions in parallel based on a reception signal according to the ultrasonic waves for detection corresponding to the detection positions from the ultrasonic probe.

20. The ultrasonic diagnosis apparatus according to claim 14, wherein
the processing circuitry displays, on a display, information indicating hardness of the tissue superposed on a B-mode image generated based on transmission of ultrasonic waves from the second transducers of the ultrasonic transducer unit for detection.
